(19) United States
(12) Patent Application Publication
(10) Pub. No.: US 2020/0190449 A1
(43) Pub. Date: Jun. 18, 2020

(54) ENRICHED ALCOHOLIC BEVERAGES

(71) Applicant: Euphoria Research and Development Ltd., Bat Yam (IL)

(72) Inventor: Daniel HAKIM, Bat Yam (IL)

(21) Appl. No.: 16/799,457

(22) Filed: Feb. 24, 2020

Related U.S. Application Data
(63) Continuation-in-part of application No. PCT/IB2018/056348, filed on Aug. 22, 2018.
Application No. PCT/IB2018/056348 is a continuation-in-part of application No. PCT/IB2019/057078, filed on Aug. 22, 2019.
(60) Provisional application No. 62/548,924, filed on Aug. 22, 2017.
(60) Provisional application No. 62/721,357, filed on Aug. 22, 2018.

(51) Int. Cl. C12G 3/024; C12G 3/005; A61K 31/198; A61K 31/4985; C12G 3/08

(52) U.S. Cl. CPC C12G 3/024; C12G 3/005; C12G 3/08; A61K 31/4985; A61K 31/198

(57) ABSTRACT

Alcoholic beverages enriched with a neurotransmitter precursor are provided. The enriched alcoholic beverages, for example, beer, wine, fruit wine or spirit, afford enhanced euphoric feeling, elevated mood, motivation, focus and sociability, which exceed similar effects exerted by corresponding non-enriched beverages, yet reduce the adverse effects associated with alcohol consumption, particularly with intoxication. Further provided are methods for enhancing and prolonging a euphoric sensation associated with alcohol consumption, and methods for enhancing a desired cognitive ability, emotion, and/or a mental skill, comprising the provision to a subject, upon alcohol consumption, of an effective amount of at least one neurotransmitter and/or at least one neurotransmitter precursor and, optionally, one or more psychostimulant substances.

### CROSS-REFERENCE TO RELATED APPLICATIONS

The present application is a continuation-in-part of International Application Number PCT/IB2018/056348, filed Aug. 22, 2018, which claims benefit of U.S. Provisional Application No. 62/548,924, filed Aug. 22, 2017. The present application is also a continuation-in-part of International Application Number PCT/IB2019/057078, filed Aug. 22, 2019, which claims benefit of U.S. Provisional Application No. 62/721,357, filed Aug. 22, 2018. The entire contents of each of the above-identified applications are hereby incorporated herein by reference.

### FIELD OF THE INVENTION

The present disclosure, in some embodiments thereof, relates to alcoholic food products, more specifically, but not exclusively to enriched alcoholic beverages. The present disclosure further relates to means and methods of elevating pleasurable effects associated with alcohol consumption and, optionally, improving cognitive abilities in a subject, for example short-term cognitive abilities.

### BACKGROUND

The element which accounts for the psychotropic effect caused by alcohol consumption is ethanol, a two-carbon chain alcohol molecule that interacts with neurotransmitter systems in the brain, and directly affects brain chemistry by altering the levels of some neurotransmitters. Ethanol is one of man's most commonly used and abused substances, but the effects on mood are paradoxical as relaxation and pleasurable effects of moderate intoxication may rapidly change to dysphoria and anxiety. Ethanol has dual properties both of a stimulant and a depressant since it affects both “excitatory” neurotransmitters and “inhibitory” neurotransmitters. A low-to-medium dose of alcohol has a depressant effect on the central nervous system. As a depressant, alcohol reduces energy levels, and slows down thought, speech and movements, exerts relaxation and drowsiness, mainly by increasing production of the inhibitory neurotransmitter gamma-aminobutyric acid (GABA), and suppressing the excitatory neurotransmitter glutamate.

As a stimulant, ethanol indirectly causes release of dopamine in the reward pathway in the nucleus accumbens (NAc) part of the brain, which accounts for euphoric feeling, cheerful mood and incentive to seek pleasure. Dopamine is released to the synapse upon alcohol consumption, and the more dopamine is released the merrier and joyful a person becomes. Re-uptake of dopamine which follows and, thereby, decreased levels of the neurotransmitter in the synapse, leads to a down feelings and depression which often characterize alcohol withdrawal or sobering up.

Reduced psychomotor functioning and cognitive abilities such as inhibition, attention control, and planning may lead to loss of control over drinking and unintentional excessive consumption. Excessive and/or prolonged alcohol consumption may have some undesired short-term physiological and psychological effects such as gastric irritation, anxiety disorders and other excitable states, and highly undesired longer-term effects such as cirrhosis, cardiomyopathy and dementia. Alcohol consumption may lead to intoxication, which, in turn, can have serious consequences such as accidents and uncontrolled violent behavior with subsequent medical complications.

The standard way alcohol is consumed is by drinking, usually over a few hours. In the case of beers/lagers, alcohol is provided as relatively dilute solution, wines are stronger, and spirits contain the maximum alcohol concentration.

One approach to reduce or abolish the undesired effects of ethanol is reducing the concentration of alcohol in drinks. Dealcoholized beverages have been known for nearly 100 years, and are disclosed, for example, in U.S. Pat. Nos. 1,390,710, 1,256,894, 6,472,009, 1,401,700, 4,999,209, and 4,612,916. While removing alcohol may seem a practical option for beers, in some cases, especially in wine and spirits, there is a marked deterioration in taste, bouquet, and other qualities of the beverage due to processes performed to eliminate or reduce alcohol content, such as boiling or steaming.

Another approach which has been practiced is the safer alcohol approach, which utilizes drugs that act in a similar way to alcohol but are free of some of its immediate adverse effects, such as gastric irritation, and do not produce the longer-term effects such as cirrhosis, cardiomyopathy and dementia. However, all the drugs had limited use due to development of dependence thereon, and/or abuse thereof.

There is an unmet need for “safer” consumption of alcoholic beverages that would not deprive the drinker from the pleasure and psychotropic and psychoactive effects provided by drinking alcohol, yet, would reduce or even be devoid of the adverse effects associated with alcohol consumption, particularly with intoxication.

### SUMMARY

The present disclosure is based on a discovery by the present inventor that the effects associated with consumption of alcoholic food products, for example, alcoholic beverages, particularly the pleasurable effects such as an euphoric perception and “high feeling”, may be enhanced and prolonged without the need to increase the amount of alcohol in the product, but merely by the concomitant consumption of a neurotransmitter precursor. Moreover, it has been discovered by the present inventor that consumption of both alcohol and at least one neurotransmitter precursor, and optionally, one or more psychostimulant substance such as caffeine, minimized and even circumvented at least some of the non-pleasurable effects associated with alcohol consumption, particularly the undesired physical and emotional sensations of intoxication.

In one aspect, the present disclosure relates to an alcoholic food product, comprising an edible base material, which may be a liquid, solid or semi-solid edible substance, for example, liquid, alcohol, and at least one neurotransmitter and/or at least one neurotransmitter precursor. The food product thus provided is an alcoholic food product, namely a food product that essentially contains a certain amount of alcohol for example, from about 0.5% to about 98% by volume or by weight. The inclusion of a neurotransmitter and/or a precursor thereof in the alcoholic food product, enriches the alcoholic food product with certain qualities that affect certain desired psychotropic and psychoactive functions and/or actions.

In some embodiments, a disclosed enriched alcoholic food product is an enriched alcoholic beverage such as beer, wine, spirit, cider, perry and alcopop.

The neurotransmitter may be, for example, norepinephrine, epinephrine, serotonin, dopamine, endorphin, acetylcholine, gamma-aminobutyric acid (GABA), and any combination thereof. The neurotransmitter precursor may be, for example, a norepinephrine precursor, for example dopamine; an epinephrine precursor; a serotonin precursor, for example, 5-hydroxytriptophan; a dopamine precursor such as L-phanylalanina, L-tyrosine or levodopa; an endorphin precursor; an acetylcholine precursor; or a GABA precursor, and any combination thereof.

In exemplary embodiments, a disclosed enriched alcoholic beverage comprises the dopamine precursor tyrosine, in amounts ranging, e.g., from about 10 mg to about 5000 mg per 1 liter, for example, from about 100 mg/L to about 1000 mg/L.

A contemplated enriched alcoholic food product, for example, enriched alcoholic beverage, may further comprise one or more psychostimulant substances, namely, a substance that is directly or indirectly involved in enhancement of a neurotransmitter synthesis and/or stability, inhibition of degradation of a neurotransmitter and/or inhibition of reuptake of a neurotransmitter. Such a psychostimulant is exemplified in some embodiments, by caffeine, wherein the amount of caffeine in a disclosed enriched alcoholic food product may be from about 10 mg/L to about 750 mg/L.

In exemplary embodiments, an enriched beer is disclosed, comprising tyrosine and caffeine, for example, enriched ale, stout, porter, or lager.

The alcoholic food product and/or the enriched alcoholic beverage, upon consumption thereof, exerts one or more positive psychoactive effects and/or one or more positive psychotropic effects which may commence 5 minutes and last up to 24 hours, or even up to 48 hours after alcohol consumption. For example, consumption of a disclosed enriched alcoholic beverage such as beer may promote enhanced and prolonged sense of euphoria.

A contemplated enriched beer, wine, spirit, alcopop and/or cider described herein may exert upon a consumer stimulant effects such as elevated mood (high spirit), energy, excitement, talkativeness, vigorous and vitality which exceed in intensity and duration similar effects exerted by the corresponding non-enriched alcoholic beverage, while reducing intoxication effects and sedative effects such as difficulty in concentrating, down feeling, heavy head, heavy body, sedative, slow thinking, and sluggishness.

The advantages of a combined consumption of both alcohol and a precursor of a neurotransmitter, which is directly or indirectly involved in a brain function that is boosted or kicked off upon alcohol consumption, are being leveraged herein by the provision of various methods for boosting, enhancing and/or improving desired or positive cognitive abilities as well as desired or positive emotions and mental functions brought upon by alcohol consumption.

Such methods are exemplified herein by the use of tyrosine, a precursor of dopamine, which is provided, in accordance with a contemplated method, together with alcohol, for example by the provision of a contemplated enriched alcoholic beverage such as beer, alcopop, wine or spirit.

In some aspects, the present disclosure relates to at least the following methods:

(1) a method for enhancing and prolonging a euphoric sensation associated with alcohol consumption, the method comprising providing to the subject, upon alcohol consumption, an effective amount of at least one neurotransmitter or a precursor thereof, thereby enhancing and prolonging a euphoric sensation in the subject.

(2) A method for enhancing or strengthening a desired emotion and/or a desired mental function in a subject brought upon, initiated, kicked off or triggered by alcohol consumption, the method comprising consuming alcohol together with one or more neurotransmitters and/or one or more neurotransmitter precursors, thereby enhancing or strengthening the desired emotion and/or mental function.

Desired emotions, desired metal functions or abilities, and desired cognitive abilities or skills are subjective emotions, mental abilities and cognitive skills, respectively, that a person wishes or eagers to feel, experience, pursue and/or practice. Desired emotions are exemplified by euphoric feeling, elevated positive and social mood, joyfulness, satisfaction, stress relief, relaxation, optimism, confidence, contempt, courage, empathy, enthusiasm, interest, passion, and self-confidence. Desired mental functions are exemplified by perception, judgement, memory, thinking, ideation, imagination, belief, reasoning, and volition.

(3) A method for ameliorating, mitigating or reducing an adverse or negative effect associated with alcohol consumption, the method comprising consuming alcohol together with one or more neurotransmitters or neurotransmitter precursors, thereby ameliorating, mitigating or reducing the adverse or negative effect of alcohol.

In some embodiments, a disclosed method may further comprise the provision, consumption or use of one or more psychostimulant as described herein, for example, caffeine

In a further aspect, the present disclosure relates to a process for the preparation of an enriched alcoholic beverage, such as beer, wine, whisky, vodka a liquor, comprising combining a base alcoholic liquid with one or more neurotransmitter precursors, and, optionally, one or more psychostimulant substances.

Unless otherwise defined, all technical and/or scientific terms used herein have the same meaning as commonly understood by one of ordinary skill in the art to which the disclosure pertains. Although methods and materials similar or equivalent to those described herein can be used in the practice or testing of embodiments of the disclosure, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE FIGURES

Some embodiments disclosed herein are described, by way of example only, with reference to the accompanying drawings. With specific reference now to the drawings in detail, it is stressed that the particulars shown are by way of example and for purposes of illustrative discussion of embodiments of the present disclosure. In this regard, the description taken with the drawings makes apparent to those skilled in the art how embodiments described herein may be practiced.

In the drawings:

FIGS. 1A-1B are bar graphs showing stimulant effects (STIM) values obtained in an exemplary biphasic alcohol effects scale (BAES) test conducted with beer enriched with tyrosine and caffeine (1B) and a corresponding non-enriched beer (1A); and

FIGS. 2A-2B are bar graphs showing sedative effects (SED) values obtained in an exemplary biphasic alcohol effects scale (BAES) test conducted with beer enriched beer with tyrosine and caffeine (1B) and a corresponding non-enriched beer (1A).

### DETAILED DESCRIPTION

The present invention, in some embodiments thereof, relates to enriched alcoholic food products, more specifically, but not exclusively to enriched alcoholic beverages. In some further embodiments, the present disclosure relates to means and methods of elevating pleasurable effects associated with alcohol consumption and, optionally, improving cognitive abilities in a subject, for example short-term cognitive abilities.

Alcohol is one of the favorite, commonly used, yet a somehow dangerous psychoactive substance. The prevailing and predominant way alcohol is consumed is by drinking alcoholic beverages, usually over a few hours. People consume alcohol for several reasons such as quenching thirst, heating or cooling the drinker, for the taste of alcohol, the association alcoholic beverages have with other aspects of life such as food and friendship, and mostly due to the psychological and psychotropic effects of alcohol associated with alcohol consumption. Consuming alcohol often confers upon the consumer a euphoric sensation or a “high” feeling, a cheerful mood, relaxation, and a reduced self-awareness. Part of the pleasure of alcohol consumption, at least for the non-dependent consumer, is the associated rituals of consumption that may fill primitive appetitive functions.

While seeking for the emotional and psychological benefits when consuming alcoholic products, there are, inherently, short term and long-term undesired consequences of consuming alcohol, more so when consuming excessive amount of alcohol.

Dealcoholized beverages, such as beer and wine, designed in attempt to address the need to reduce the hazardous consequences of alcohol intoxication, are available in reasonably palatable forms. However, while non-alcoholic drinks can fill such use-values of alcoholic beverages as quenching thirst and heating or cooling the drinker, they do not provide the range of psychoactivity and palatability which people are seeking in alcohol.

Unless stated otherwise, “alcohol” as referred to herein is ethanol.

The present disclosure is based on a discovery by the present inventor that the effects associated with consumption of alcoholic food products, for example, alcoholic beverages, that contain a neurotransmitter precursor, particularly the pleasurable effects, may be enhanced and prolonged without the need to increase the amount of alcohol in the product. Moreover, the present inventor envisaged and successfully obtained alcoholic beverages containing at least one neurotransmitter and/or a precursor thereof, and optionally further containing a psychostimulant substances such as caffeine, which provide to the consumer stronger and continuing euphoric feeling while minimizing and even circumventing at least some of the non-pleasurable effects associated with alcohol consumption, particularly the undesired physical and emotional sensations of intoxication.

Disclosed herein is a discovery by the present inventor that consumption of alcoholic food products containing neurotransmitter precursors affects the level of certain neurotransmitters in the brain, for example neurotransmitters that are boosted or released due to alcohol consumption. Alteration, for example, elevation, of certain brain neurotransmitters level, increases or enhances, e.g., the euphoric sensation provided or bestowed by alcohol. This euphoric feeling lasts longer than that induced by consuming alcohol alone, and, furthermore, it is mostly not accompanied by undesired physiological and psychological short-term effects associated with the concomitant and rather intense decrease in neurotransmitters level that follows shortly afterward.

In an aspect of the disclosure, there is provided a food product comprising an edible base material, alcohol and one or more neurotransmitters and/or neurotransmitter precursors.

The term “food product” as used herein refers to a substance that can be used or prepared for use as food by humans or non-humans.

“Alcoholic food product” (or “alcoholic product”) is a composition of an edible base material defined herein as any substance composed of carbohydrates, fats, proteins and/or water, and alcohol (e.g., ethanol) in the amount of 0-99%, for example from about 1% to about 20%, from about 1% to about 5%, from about 3% to about 8%, from about 5% to about 10%, from about 5% to about 15%, from about 8% to about 12%, from about 10% to about 15%, from about 15% to about 25%, from about 20% to about 30%, from about 25% to about 40%, from about 30% to about 50%, from about 40% to about 60%, from about 50% to about 65%, or from about 60% to about 80%, ethanol by weight or by volume. It can be eaten or drunk by humans or by animal for nutrition or pleasure.

Non-limiting examples of alcoholic food products include, alcoholic liquids comprising a base material such as water, juice, e.g., fruit or vegetable juice, and milk and alcohol. Alcoholic liquid products include, for example, beverages such as beer, wine, spirit and the like. Solid or semi-solid alcoholic products are edible foods that contain alcohol, for example, foods that have been cooked, combined and/or mixed with alcohol. Such alcohol-containing solid or semi-solid products are exemplified by meat, fish, chicken, fruits, breads, soups, stews, sauces, fondues, backed desserts such as cakes, pastries such as cookies and pies, and no-bake desserts such as creams, ice-cream, puddings and mousses, extract flavoring such as pure vanilla extract and pure almond extract, filled candies such as filled chocolate, snacks and food flamed with alcohol.

In some embodiments, a contemplated alcoholic food product is an alcoholic beverage.

The terms “enriched alcoholic food product” and “enriched alcoholic beverage” as used herein refer to an alcoholic food product or alcoholic beverage, respectively, enriched by the addition thereto, provision thereto, mixing thereof, or combining it with at least one neurotransmitter and/or at least one neurotransmitter precursor. “Enrichment” of the alcoholic food products or alcoholic beverages is further meant herein enriching the product with desired qualities imparted to it by enriching it with at least one neurotransmitter and/or a precursor thereof.

The term “one or more neurotransmitter precursors” as used herein is interchangeable with the term “at least one neurotransmitter precursor” and means at least one precursor of at least one neurotransmitter.

The action of alcohol is biphasic: when blood alcohol concentration (BAC) levels are rising, the stimulant properties of alcohol are more pronounced; when BAC levels are falling, the depressant effects of alcohol are more pronounced, Ethanol, being a small molecule, can interact with many neurotransmitter systems in the brain. It directly affects brain chemistry by altering levels of neurotransmitters. Alcohol affects both “excitatory” neurotransmitters and “inhibitory” neurotransmitters and, hence, has the properties of both a stimulant and a depressant. This makes the action of alcohol in the brain very different from, and much more complex than, large molecules such as opiates, tetrahydrocannabinol (THC), or amphetamine, which simulate a specific neurotransmitter and interact with a specific neurotransmitter system.

### Neurotransmitters and Precursors Thereof

Neurotransmitters play a key role in the function of the central nervous system, being chemical “messengers” that transmit signals throughout the body, signals such as those controlling thinking processes, behavior and emotion. Neurotransmitters are excreted to, and travel through, the synapses, i.e., small gaps between the axon terminal of neurons that release neurotransmitters in response to an impulse, and the membrane of adjacent axons, dendrites, muscle or gland cells having the appropriate receptors for binding the neurotransmitters. Neurotransmitters can either prompt or suppress the further signaling of nearby neurons. The main neurotransmitters include norepinephrine, epinephrine, serotonin, dopamine, endorphin, acetylcholine, gamma-aminobutyric acid (GABA), glycine, glutamic acid, aspartic acid, and taurine, the first six of which are neurotransmitters synthesized from amino acids, and the last three are amino acids per se.

The term “neurotransmitter precursor”, as used herein, refers to a substance that can be converted into a neurotransmitter in the body, particularly in the brain, usually through enzymatic reactions such as metabolic processes.

Neurotransmitters which may be directly or indirectly affected by alcohol include GABA, endorphins, glutamate, dopamine, norepinephrine and adrenaline (epinephrine).

Gamma-aminobutyric acid (GABA) is an inhibitory neurotransmitter, which reduces energy levels and calms everything down. Drugs like Xanax and Valium increase GABA production in the brain, resulting in sedation. Alcohol increases the effects of GABA and affects the GABA system in a manner similar to valium leading to relaxation and drowsiness. Alcohol's effect on the GABAnergic system may also be responsible for its depressant effects.

Glutamate is an excitatory neurotransmitter which normally increases brain activity and energy levels. Alcohol suppresses glutamate levels probably by inhibiting the N-methyl-d-aspartate (NMDA) glutamate receptor, resulting in a slowdown along the brain's highways. It is alcohol's effects on the glutamate system which lead to staggering, slurred speech, and memory blackouts.

Endorphins are produced in response to certain stimuli, especially stress, fear or pain. They originate in various parts of the body such as the pituitary gland, spinal cord and throughout other parts of the brain and nervous system and interact mainly with receptors in cells found in regions of the brain responsible for blocking pain and controlling emotion. Alcohol affects the endorphin system in a manner similar to opiates, acting as a painkiller and giving an endorphin “high”.

Adrenaline (epinephrine) and its precursor norepinephrine (noradrenalin), are important hormones produced by the adrenal gland. They increase the heart rate, blood pressure, and blood glucose levels, open up the airways in the lungs and, like cortisol, help inhibit non-essential bodily functions. Alcohol causes the adrenal glands to release adrenaline and norepinephrine which ultimately enter the brain and promote alertness and “fight response”. This is one reason why alcohol acts as stimulant.

Alcohol increases release of dopamine (DA). Dopamine, 3,4-dihydroxyphenethylamine, is an organic chemical of the catecholamine and phenethylamine families having the chemical structure:

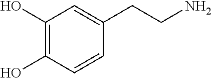 [SMILES: NCCc1ccc(O)c(O)c1]

This amine is synthesized by removing a carboxyl group from its precursor levodopa (L-dopa; dihydroxyphenylalanine), which is synthesized in the brain and kidneys during the metabolism of the amino acid tyrosine. It is a neurotransmitter in itself, and a precursor of the hormone neurotransmitters epinephrine and norepinephrine. Two main brain areas produce dopamine that relays signals that travel throughout the brain: the substantia nigra, a tiny strip of tissue on either side of the base of the human brain (situated in a region known as the midbrain), and the close by ventral tegmental area (VTA). Dopamine from the substantia nigra helps in regulating movements and speech, thus, helping in regulating emotional responses.

The reward center (also known as the “reward pathway”) is comprised primarily of the nucleus accumbens (NAc), the VTA, and a part of the prefrontal cortex. The reward center is affected by virtually all pleasurable activity, including everything from hanging out with friends, going on vacation, ingesting drugs (e.g., cocaine), listening to music, eating and drinking, having sexual activity, and consuming alcohol. The ventral tegmental area usually sends dopamine into the brain when animals or humans expect or receive a reward, and helps control the brain's reward and pleasure centers. All things which give us pleasure, cause a release of dopamine in the reward pathway as well as trigger a number of other events in the brain including endorphin release and activation of the orbitofrontal region of the prefrontal cortex.

Dopamine is known as the “motivation molecule” or the “reinforcement molecule” as it is responsible for reward-seeking behavior and helps provide the drive, focus and concentration needed to get things done. This dopamine release tells the brain that whatever it just experienced is worth getting more of, and that helps animals and humans change their behaviors in ways that will help them attain more of the rewarding item or experience. This brain reward system is associated with “feeling good” and promotes survival of the species by rewarding behaviors necessary for continued survival.

Dopamine is also involved in many other functions of the brain including motor activity, motivation, learning, pain processing, mood, attention span and regulation of sleep. It regulates stress relief.

Alcohol consumption triggers a boost of dopamine release in the reward center, leading to the relaxing and carefree experience of the alcohol “buzz”. Consumption of even small amounts of alcohol increases the amount of dopamine in the NAc in a dose-response manner. Alcohol increases dopamine via the promotion of synaptic terminal dopamine release rather than via the inhibition of dopamine transporters. Alcohol can also indirectly increase DA levels by affecting the GABA system and the endorphin system. Neurons from the GABA system extend into the reward pathway and when alcohol affects the GABA system these neurons release dopamine into the reward pathway. Likewise, neurons extend from the endorphin system into the reward pathway and these also release dopamine into the reward pathway when alcohol directly stimulates the endorphin system.

A boost in the activation of dopaminergic pathways eventually causes a depletion of the dopamine reserves within the brain cells, leaving the brain in an imbalanced state. As dopamine itself is impenetrable to the blood-brain barrier, it must be synthesized in situ from its precursors. Over time, with more drinking, the stimulating effect of dopamine diminishes until it's almost nonexistent.

It has been postulated by the present inventor that kicking-off brain dopamine release via alcohol consumption, and simultaneously providing means to sustain elevated brain dopamine level by providing brain permeable dopamine building blocks, may enhance the euphoric feeling and afford qualities such as increased motivation, focus and concentration to pursue rewarding actions such as working and learning, increased sociability, and attention, while at the same time alleviating alcohol withdrawal symptoms and, moreover, substantially decreasing addiction disposition.

The present inventors envisaged, and successfully produced, alcoholic food products such as alcoholic beverages, containing a precursor of dopamine, for example, tyrosine, and, optionally, a psychostimulant substance such as caffeine, relying on the strong affiliations of these ingredients to the metabolism of dopamine within the brain, thereby, assumingly, promoting dopamine synthesis within the central nervous system and establishing a long lasting steady state of readily available dopamine.

In some embodiments, the alcoholic products described herein are designed to promote at least the levels of dopamine in the brain. To this end, the alcoholic products are provided with one or more dopamine precursors. For example, L-phenylalanine (Phe), L-tyrosine (Tyr) and/or levodopa (L-dopa) are the precursors of dopamine as well as other catecholamines such as norepinephrine and epinephrine.

The terms “dopamine precursor” and “dopamine immediate metabolic precursor” as used herein are interchangeable and refer to a substance that can be converted into dopamine in the body through a series of one or more metabolic reactions. The primary and minor metabolic pathways for obtaining dopamine (DA) from its precursors are:

Primary: phenylalanine (Phe)→tyrosine (Tyr)→levodopa (L-dopa)→DA

Minor: Phe→Tyr→p-Tyramine→DA

Minor: Phe→m-Tyr→m-Tyramine→DA

The direct or immediate metabolic precursor of dopamine, L-dopa, can be synthesized indirectly from the essential amino acid Phe or directly from the non-essential amino acid Tyr. L-Phenylalanine is converted into Tyr by the enzyme phenylalanine hydroxylase, with molecular oxygen (O₂) and tetrahydrobiopterin (THB) as cofactors. L-Tyrosine is converted into L-dopa by the enzyme tyrosine hydroxylase, with tetrahydrobiopterin, O₂, and iron (Fe²⁺) as cofactors. L-dopa is converted into dopamine by the enzyme aromatic L-amino acid decarboxylase (also known as dopa decarboxylase), with pyridoxal phosphate (the active form of vitamin B₆) as the cofactor.

In some embodiments, a contemplated alcoholic food product supplemented with Tyr may increase DA brain level. In some embodiments, a contemplated alcoholic product may afford sustained elevated DA levels in the brain initiated by alcohol consumption, to thereby provide at least some of the desired benefits exerted by dopamine release, for example joyful and euphoric feeling, and relaxation.

Without wishing to be bound by theory, it is assumed that when a contemplated alcoholic food product, e.g., an alcoholic beverage, is consumed, the alcohol in the product causes an immediate release of dopamine. A dopamine precursor contained in the product provides sustained supply of dopamine building blocks from the very first moment of alcohol intake and reinforces the neurotransmitter supply in the brain. Thereby dopamine levels in the brain are maintained or even amplified long after the alcohol level in the blood has gown down to zero, affording not only a prolonged euphoric feeling but, moreover, mitigating, quenching and/or circumventing the unpleasant psychological and physiological sensations associated with alcohol withdrawal such as headache, nausea and depression. These effects outgo the psychotropic effects associated with consumption of high amounts of alcohol alone.

In some embodiments, supplementing an alcoholic food product, for example, alcoholic beverage such as beer or wine, with precursors of neurotransmitters, bestows to it the ability to impart upon a consumer thereof at least the following positive psychoactive effects: blissful, joyful and euphoric sensation. The term “psychoactive” as used herein means affecting the mood, and “positive psychoactive effect” herein means affect the mood is a positive, desired manner.

Positive psychoactive effects, e.g., euphoria, exerted by a contemplated alcoholic food product may sustain long after consumption of the product, and it may not be accompanied by drunkenness or intoxication. In some embodiments, a euphoric feeling may last from about 5 minutes up to 24 hours post consumption, with almost no accompanying feeling of intoxication. For example, a euphoric state of mind may last about 5-10 min, about 10-15 min, about 10-20 min, about 20-30 min, about 30-40 min, about 40-50 min, 0-1 hours, about 1-3 hours, about 1-4 hours, about 2-5 hours, about 3-5 hours, about 4-6 hours, about 5-8 hours, about 5-9 hours, about 6-10 hours, about 7-10 hours, about 8-11 hours, about 9-10 hours, about 9-11 hours, about 9-15 hours, about 10-12 hours, about 12-15 hours, about 15-18 hours, about 15-20 hours, about 18-22, about 20-24 hours, or even longer, after blood alcohol level has already dropped to zero.

Often, upon consumption of alcohol, speech and movements of the person consuming alcohol are slowed down, and the more alcohol is consumed the more of these effects are felt. Alcohol directly affects the cerebral cortex, namely, the region in the brain where thought processing and consciousness are centered. Alcohol can depress the behavioral inhibitory centers, making the person less inhibited; it slows down the processing of information from the eyes, ears, mouth and other senses; and it inhibits the thought processes, making it difficult to think clearly. Alcohol also affects the cerebellum, the center of movement and balance in the brain, resulting in the staggering, off-balance swagger often associated with the so-called “falling-down drunk.”

One of the unique and unexpected properties of a contemplated alcoholic food product provided herein, e.g., an enriched alcoholic beverage, is that it does not dim the consumer's sense of alertness as often happens upon alcohol consumption, but to contrary: it keeps the consumer well and sharply focused, fully conscious, in-control and with the ability to remain concentrated for extended periods, contrary to the psychotropic effects exerted by regular alcoholic products particularly alcoholic beverages. In fact, a contemplated alcoholic food product affords to a consumer thereof better and prolonged focusing and concentration abilities in spite the alcohol it contains.

The term “psychotropic”, as used herein, means the ability to influence the mind or cognitive abilities of a person, and/or to affect mental processes (e.g., emotions, perception) and mental activity (e.g., behavior). The term “positive psychotropic effect” herein means affect the mind, mental and/or the cognitive abilities is a positive, desired manner.

In some embodiments, supplementing an alcoholic food product, for example, alcoholic beverage such as beer or wine, with one or more neurotransmitters and/or one or more precursors of neurotransmitters as described herein, bestows the alcoholic food product the ability to exert one or more positive psychotropic effects which may last hours and even days after consumption thereof. In some embodiments, a positive psychotropic effect, for example, higher motivation, or improved ability, e.g., to concentrate and maintain focused, alert, awaken, self-confident and/or improved sociability lasts from 10 minutes to about 72 hours after consuming the enriched alcoholic food product.

For example, a person consuming, e.g., an enriched alcoholic beverage such as beer or wine may experience higher motivation to conduct rewarding activities, improved sociability, better cognitive, mental and/or emotional function, and/or physical sense of well-being, from about 5 min to about 10 min, from about 8 min to about 15 min, from about 10 min to about 20 min, from about 25 min to about 40 min, from about 0.5 hour to about 2 hours, from about 1.5 hours to about 4 hours, from about 2 hours to about 5 hours, from about 3 hours to about 6 hours, from about 4 hours to about 7 hours, from about 4 hours to about 8 hours, from about 5 hours to about 8 hours, from about 6 hours to about 9 hours, from about 6 hours to about 10 hours, from about 7 hours to about 12 hours, from about 9 hours to about 15 hours, from about 8 hours to about 16 hours, from about 10 hours to about 15 hours, from about 15 hours to about 20 hours, from about 18 hours to about 22 hours, from about 20 hours to about 25 hours, from about 25 hours to about 35 hours, or even from about 35 hours to about 48 hours, after consuming the beer or wine enriched with one or more neurotransmitter and/or precursors thereof.

In some embodiments, a person consuming, e.g., a disclosed enriched alcoholic beverage such as beer, wine or spirit may experience higher motivation, better or improved creative thinking, concentration, wakefulness, self-confident and/or joyful self-contentment that may last from about 0.25 day to about 0.5 day, from about 0.5 day to about 0.75 day, from about 0.5 day to about 1.0 day, from about 0.5 day to about 1.25 days, from about 0.75 day to about 1.0 day, from about 1.0 day to about 1.5 days, from about 1.5 days to about 2.0 days, from about 1.5 days to about 2.5 days, from about 2.0 days to about 2.5 days, or from about 2.5 days to about 3.0 days, and even longer, after consuming the beer, wine, fruit wine or spirit enriched with one or more neurotransmitter and/or precursors thereof.

For example, elevated levels of DA conferred or induced by a contemplated enriched alcoholic food product may enhance the expectation of pleasure in a person consuming the product.

A contemplated alcoholic food product enriched, for example, with one or more DA precursors, for example Tyr, may positively affect memory and learning, as dopamine activity in the brain plays a substantial part in memory and learning. It is essential for long-term memory storage and retrieval. Dopamine further signals important events: it helps remembering events that have motivational significance. This ensures that memories are relevant and accessible for future behavior. Dopamine also plays an essential role in working memory. Working memory is the capacity to use information from short-term memory for guiding one's own actions.

For example, a contemplated alcoholic food product enriched with one or more DA precursors may positively affect focus and attention. Moderate levels of dopamine (not too high or too low) improve the capacity of individuals to switch attention efficiently between tasks. Furthermore, moderate levels of dopamine direct attention more efficiently to stimuli that are relevant to ongoing tasks.

For example, a contemplated alcoholic food product enriched with one or more DA precursors may positively affect pathways associated with social and extroversion behavior and/or with forming romantic attachments. Intense romantic love is associated with the dopamine reward system, and the crosstalk between oxytocin, the “love molecule”, and dopamine.

A contemplated alcoholic product enriched with one or more DA precursors may further confer or bestow to the consumer one or more of the following benefits or effects:

(a) controlled sleep-wake cycle. Dopamine D1 receptor (DRD1) activation induces arousal and wakefulness;

(b) increased creativity. Dopamine is involved in cognitive flexibility—one of the main components of creativity and creative thinking. Dopamine is also responsible for openness to new experiences, another factor associated with creativity;

(c) stimulated sexual drive. A person's response to sex, just like other rewards, is largely dependent on DA. Erections are dependent upon activation of both dopaminergic neurons (ventral tegmental area) and dopamine receptors (NAc); and

(d) depression amelioration. Elevated DA levels exerted by a disclosed product may ameliorate mental conditions related to low DA levels, such as hopelessness, worthlessness, stress handling, lack of interest in life, decreased motivation, procrastination, inability to feel pleasure, altered sleep patterns, mood swings, and impulsive or self-destructive behaviors.

In exemplary embodiments the neurotransmitter precursor or the DA precursor of a contemplated enriched alcoholic food product is L-tyrosine (Tyr).

In some embodiments, the enriched alcoholic product is an alcoholic beverage supplemented with at least one neurotransmitter precursors, which is a DA precursor selected from Phe, Tyr or L-dopa.

In exemplary embodiments, the dopamine precursor supplemented to the beverage is Tyr.

The amounts of tyrosine provided to the alcoholic beverages described herein is determined depending on some variables, for example, the amount of alcohol in the beverage, the amount and type of other supplements in the alcoholic beverage that may cross react with tyrosine, and the amount and type of other supplements provided to the beverages that may further promote elevated levels of dopamine. Usually, the amount of Tyr is in the range of 10 mg to 5000 mg per 1 liter of beverage, for example, from about 10 mg to about 30 mg, from about 20 mg to about 50 mg, from about 30 mg to about 60 mg, from about 50 mg to about 70 mg, from about 50 mg to about 100 mg, from about 125 mg to about 145 mg, from about 130 mg to about 150 mg, from about 150 mg to about 200 mg, from about 100 mg to about 250 mg, from about 200 mg to about 400 mg, from about 200 mg to about 300 mg, from about 200 mg to about 500 mg, from about 300 mg to about 400 mg, from about 300 mg to about 700 mg, from about 400 mg to about 800 mg, from about 500 mg to about 900 mg, from about 500 mg to about 1000 mg, from about 800 mg to about 1000 mg, from about 900 mg to about 1100 mg, from about 1000 mg to about 1200 mg, from about 1000 mg to about 1500 mg, from about 1000 mg to about 1600 mg, from about 1500 mg to about 2000 mg, from about 2000 mg to about 3000 mg, from about 3000 mg to about 4000 mg, or from about 4000 mg to about 5000 mg per liter, and any subranges and individual values therebetween.

In exemplary embodiments, the amount of tyrosine is about 350 mg/L, about 500 mg/L, about 750 mg/L, or from about 100 mg/L to about 1000 mg/L.

### Psychostimulant Substances

The enriched alcoholic food products described herein may further comprise supplements that promote or support increased level of a neurotransmitter such as, but not limited to, norepinephrine, serotonin, dopamine, endorphin, acetylcholine, GABA, glycine, glutamic acid, aspartic acid, and/or taurine.

The terms “psychostimulant supplement” and “psychostimulant substance”, herein used interchangeably, refer to an edible substance that has a direct or indirect effect in increasing the levels of one or more neurotransmitters in the body, particularly in the brain, or in maintaining elevated neurotransmitter levels brought about, for example, by in situ conversion of a neurotransmitter precursor into a neurotransmitter, and/or by consuming alcohol. A psychostimulant substance may be directly or indirectly involved in enhancement of neurotransmitter synthesis and/or neurotransmitter stability, or upregulation of a neurotransmitter, for example, by upregulating enzymes that synthesize the neurotransmitter or protect the neurotransmitter from metabolic degradation, for example, by inhibiting or down regulating a neurotransmitter metabolic enzyme. A psychostimulant substance may, additionally or alternatively, be directly or indirectly involved in inhibition of neurotransmitter reuptake. Psychostimulant substances may be, for example, foods which supply building blocks for production of a neurotransmitter or a precursor thereof.

In some embodiments, a psychostimulant substance increases metabolism rate in the body, thereby increasing metabolic conversion of a neurotransmitter precursor into a neurotransmitter.

In some embodiments, a psychostimulant substance inhibits or blocks metabolic enzymes that degrade a neurotransmitter precursor in the blood stream or in the digestive system.

Non-limiting examples of psychostimulant substances include caffeine, omega-3, fatty acids such as docosahexaenoic acid (DHA), magnesium, soluble fibers, folate, olive oil or monounsaturated fats extracted therefrom, green tea or theanine extracted therefrom, pregnenolone and any derivative thereof, uridine, iron, spices such as turmeric or curcumin extracted therefrom, Rhodiola rosea or an extract thereof, oregano or an extract thereof, co-factors, vitamins such as vitamin C and vitamin B6, minerals and the like.

Caffeine is the most widely consumed psychostimulant substance. It is known to highly effects the metabolism within the central nervous system via the blockade of adenosine receptors, which modulate the neurotransmission of glutamate, serotonin, acetylcholine and dopamine. Caffeine itself has a wide variety of effects on the dopaminergic system, which is crucial for the expression of caffeine's stimulating properties. Furthermore, caffeine promotes tyrosine hydroxylase activation via cellular Ca²⁺ entry stimulation mechanism, and it has been shown that chronic caffeine intake increases tyrosine hydroxylase mRNA expression. As tyrosine hydroxylase is the rate-limiting enzyme in the biosynthesis of DA and other catecholamines, caffeine may accelerate DA synthesis through upregulation of this enzyme. Caffeine may provide an energy boost just like sugar and alcohol. However, caffeine alone indirectly promotes DA level elevation only temporarily.

Without wishing to be limited by theory, it is assumed that providing caffeine and alcohol, together with a supply of DA precursors such as tyrosine, phenylalanine and/or any other DA precursor, may enable enhanced and continued conversion of immediate metabolic DA precursors to DA, thus affording stable elevated levels of DA in the brain for a prolonged time, hence a prolonged duration of desired psychotropic and/or psychoactive effects such as euphoric and bliss feelings, high motivation and energy, that would last long after consumption of alcohol ceased.

In an enrich alcoholic food product, for example, an enriched alcoholic beverage, the amount of caffeine may be in a range of from about 0 mg to about 2 gr per 1 liter of beverage. For example, from about 5 mg/L to about 10 mg/L, from about 10 mg/L to about 20 mg/L, from about 10 mg/L to about 50 mg/L, from about 00 mg/L to about 80 mg/L, from about 60 mg/L to about 100 mg/L, from about 80 mg/L to about 120 mg/L, from about 100 mg/L to about 150 mg/L, from about 120 mg/L to about 148 mg/L, from about 150 mg/L to about 200 mg/L, from about 200 mg/L to about 300 mg/L, from about 210 mg/L to about 250 mg/L, from about 200 mg/L to about 400 mg/L, from about 300 mg/L to about 500 mg/L, from about 500 mg/L to about 800 mg/L, from about 600 mg/L to 900 mg/L, from about 700 mg/L to 750 mg/L, from about 800 mg/L to about 1000 mg/L, from about 800 mg/L to about 1200 mg/L, from about 900 mg/L to about 1200 mg/L, from about 1100 mg/L to about 1400 mg/L, from about 1200 mg/L to about 1500 mg/L, from about 1500 mg/L to about 1700 mg/L, from about 1500 mg/L to about 2000 mg/L, or from about 1700 mg/L to about 2000 mg/L, and any subranges and individual values therebetween.

In certain embodiments, liquid alcoholic food products described herein contain from about 10 mg/L to about 1200 mg/L, for example, about 35 mg/L about 50 mg/L, about 100 mg/L or 350 mg/L of caffeine.

In some embodiment an enriched beer is provided comprising a DA precursor such as tyrosine and a psychostimulant substance such as caffeine.

A contemplated enriched beer, for example, can contain Tyr and about 0.75 mg/ml caffeine and yet maintain the original taste, aroma, palatability and gas content of a beer, with no hint for caffeine's dominant bitter taste.

Further psychostimulant substances suitable for the purpose of maintaining and/or increasing dopamine brain levels include vitamins and certain minerals such as, but not limited to, zinc, vitamin B6, folate, vitamins C and E, and magnesium.

A further psychostimulant substance useful is embodiments described herein is pregnenolone. Pregnenolone is the main steroid produced from cholesterol mainly in the brain, gonads and adrenal glands. Pregnenolone and its sulfate (pregnenolone sulfate) are excitatory neurosteroids, i.e., they stimulate the brain, and can increase DA. Pregnenolone and/or its derivatives have anti-stress and mood-elevating effect, they enhance learning and memory and increase the amount of deep sleep, improve energy, vision, clarity of thinking, wellbeing, and often sexual enjoyment or libido.

The amount of pregnenolone and derivatives thereof, particularly pregnenolone sulfate, in, e.g., an alcoholic beverage described herein, may be in the range of from about 0 mg to 3.0 mg per liter, for example 0.1-0.5 mg/L, or about 1 mg/L.

Psychostimulant substances are further exemplified herein by curcumin, theanine, and Rhodiola extract. Curcumin is known to be the most active phytochemical in the yellow dietary spice turmeric. Curcumin has been proven to have antioxidant, anti-inflammatory, anti-microbial, hypoglycemic, anti-rheumatic, wound healing and anti-cancer activities. Curcumin further possesses antidepressant properties by way of interacting with dopamine receptors and increasing brain dopamine levels. For example, curcumin increases DA concentration in the brain by inhibiting monoamine oxidase (MAO)-mediated DA break down. Curcumin may be taken daily in large amounts, even up to 8 gr/day.

The amount of curcumin in, e.g., a beverage described herein, may be in the range of from about 0.1 mg/ml to 2.0 g/ml, for example from about 0.2 to about 0.5 g/ml.

L-theanine is an amino acid uniquely found in green tea that creates an alert state of relaxation without drowsiness. L-theanine is known to be able to cross the blood-brain barrier and increase dopamine levels in the brain. As such, it may have anti-depressant and anti-anxiety effects, it may reduce mental and physical stress, and lead to improvements in learning and memory in humans and animals. Even just a single, small dose of L-theanine (100 mg) significantly improves the ability to pay attention and maintain focus.

The amount L-theanine e.g., in an alcoholic beverage contemplated herein, may be in the range of from about 0 mg to about 2000 mg per 1 liter of beverage, for example, from about 10 mg/L to about 30 mg/L, 20 mg/L to about 50 mg/L, 50 mg/L to about 100 mg/L, 80 mg/L to about 150 mg/L, 100 mg/L to about 150 mg/L, 150 mg/L to about 200 mg/L, 150 mg/L to about 400 mg/L, 300 mg/L to about 500 mg/L, 550 mg/L to about 750 mg/L, 500 mg/L to about 1000 mg/L, 700 mg/L to about 1200 mg/L, 1100 mg/L to about 1250 mg/L, 1200 mg/L to about 1500 mg/L, 1550 mg/L to about 1850 mg/L, or 1700 mg/L to about 1000 mg/L.

In some embodiments, enriched alcoholic beverages described herein contain 600 mg/L, 1200 mg/L or 1800 mg/L of L-theanine.

Rhodiola rosea, or “golden root,” is a popular plant in traditional medicine in Eastern Europe and Asia, with a reputation for improving depression, enhancing work performance, eliminating fatigue and treating symptoms resulting from intense physical and psychological stress. Rhodiola exerts its benefits via multiple effects on the central nervous system, including enhancing the stability of dopamine. This leads to notable decreases in depression, anxiety, and fatigue, as well as an increased ability to handle stress. Rhodiola extract derived from Rhodiola rosea root and standardized to contain 3% total rosavins and a minimum of 1% salidrosides may be included in, e.g., enriched alcoholic beverage described herein in amounts which range from about 300 mg to about 2000 mg per liter, for example, 510 mg/L, 800 mg/L or 1100 mg/L.

Other psychostimulant substances which may find use in embodiments described herein include, for example, nutritional or brewer's yeasts which is rich in uridine-5w-monophosphate that may increase DA levels in the brain; oregano, which increases DA levels by decreasing DA break down and reuptake; and resistant starch, a type of soluble fiber that increases butyrate, which may increase dopamine levels.

In some embodiment, a disclosed enriched alcoholic food product comprises one or more psychostimulant substances that directly or indirectly affect DA brain level, for example, caffeine, theanine, curcumin, uridine, pregnenolone, and/or oregano.

Contemplated alcoholic food products such as beverages enriched with a dopamine precursor such as tyrosine and further comprising a psychostimulant substance such as caffeine or theanine, provide the exact combination of alcohol, DA precursor and psychostimulant substance that affords an optimal rate of brain dopamine level increase, as well as optimal, stable DA level that provide to the consumer intensified, long lasting pleasure and a pleasant drinking experience, while circumventing the “down” feeling often associated with alcohol consumption.

The amounts of psychostimulant substance provided to the enrich alcoholic products described herein vary and depend on the psychostimulant substance itself as well an on the other ingredients in the enriched alcoholic product, particularly the amount of alcohol, the amount and type of DA precursor(s), the presence of other neurotransmitter or precursors thereof, and/or the amount and type of other supplements that may cross react or enhance the effect of a particular psychostimulant substance. Excess dopamine is dangerous and needs to be avoided, and a skilled person would appreciate that the amount and number of psychostimulant substances provided to the alcoholic beverages are to be adjusted so as to avoid excess and undesired DA levels.

The amount of one or more psychostimulant substances in a contemplated enriched alcoholic food product may be in a range of from 0 to about 10% by weight. For example, from about 0.01% to about 1.00%, from about 0.5% to about 1.00%, from about 0.1% to about 0.3%, from about 0.2% to about 0.5%, from about 0.5% to about 1.0%, from about 1.0% to about 3.0%, from about 1.0% to about 5.0%, from about 5.0% to about 10.0%, from about 8% to about 10.0%, by weight or by volume of total enriched alcoholic product, and any sub-ranges and/or individual values therebetween.

The amount of psychostimulant substances in a contemplated product are adjusted such that the original texture, smell and palatability of the product is maintained.

### Alcoholic Beverages

In one aspect, the present disclosure relates to an alcoholic beverage comprising a base liquid, alcohol, one or more neurotransmitter and/or one or more neurotransmitter precursors as defined herein, and, optionally, further comprising one or more psychostimulant substances as defined herein. In exemplary embodiments, the neurotransmitter precursor is a dopamine precursor and the optional psychostimulant substance is caffeine.

Herein throughout, the term “base liquid” describes a liquid form of a substance or a mixture of substances which either alone or when mixed with other additives can form a beverage. In some embodiments, the base liquid is a base beverage.

In some embodiments, the base beverage is an alcohol-free base beverage.

The phrase “alcohol-free base beverage”, as used herein, is a beverage having alcohol percentage that is no more than 50% of the alcohol content in a corresponding alcoholic beverage, preferably no more than 40%, no more than 30%, no more than 20%, no more than 15%, no more than 10%, no more than 5%, no more than 1%, no more than 0.5%, no more than 0.1%, no more than 0.05%, or no more than 0.01%, of the alcohol content acceptable for a certain beverage, including any subranges and any intermediate values there between. This phrase is used herein as encompassing both base beverages that are typically used for forming alcoholic beverages, and base beverages which are typically used as non-alcoholic beverages, for example, juices.

In some embodiments, the alcohol-free base beverage is devoid of alcohol.

By “devoid of alcohol” is meant herein less than 0.01% or less than 0.005%, or less than 0.001%, of alcohol by volumes, or even null.

Exemplary alcohol-free base beverages which form the liquid base for the enriched alcoholic beverages described herein include, but are not limited to, natural or artificially flavored fruit juice (such as grape, mango, elder, apple, orange juice, and the like), vegetable juice, fruit syrup, concentrate or nectar from fruits, plant materials such as agave, jello, carbonated beverages such as cola, optionally with addition of roasted malt beer, caffeinated beverages, specialized flavor formulations emulating the taste of existing wines and spirits, non-alcoholic cocktails (“mocktails”), malt beer, dealcoholized ciders, dealcoholized wines, dealcoholized beers, dealcoholized spirits, tonic water and water.

When the liquid base comprises alcohol, it is termed herein a “base alcoholic liquid”. In some embodiments, the base alcoholic liquid is an alcoholic beverage. The term “alcoholic beverage” as used herein encompasses any beverage having an alcohol (ethanol) content of at least 2% by volume, whether distilled, fortified, brewed, or produced by fermentation, and includes, but is not limited to, wine, beer, fermented liquids derived in whole or in part from fruit juices, such as fruit wines, cider or perry (pear cider), spirits, flavored alcoholic beverages collectively termed herein and in the art as “alcopops”, and the like.

In some embodiments, the base alcoholic liquid is an alcoholic beverage substitute that has residual alcohol content of 0-20% by volume, depending on the alcoholic beverage being substituted.

In some embodiments, the base alcoholic liquid is a fermented base material, also referred to herein as “must”.

The alcohol content of a fermented must is about 7% to 9%, however, under certain fermentation conditions, for example, by adding sugar to the must, following a first fermentation cycle, the natural alcohol content may be in the range of 10% to 20%.

The terms “fermenting base material” and “fermentable base material”, as used herein, are interchangeable and refer to any sugar-containing mash, juice, sap, or extract produced form a plant material such as, but not limited to, fruit, berries, grains, vegetables, sugarcane, sugar palm, cactus plant or sap tapped from trees, which can be used in a fermentation process. Fermentable base material, as used herein, further encompasses honey, specialized flavor formulations and any other natural or non-natural sugar containing edible material.

The term “fermented base material”, as used herein, is a material produced or obtained from a fermentable base material, as defined herein, at the end of a fermentation process.

In some embodiments, the fermented base material is produced from one or more natural or artificially flavored fruit juice, berry juice, vegetable juice, fruit syrup, concentrate or nectar from fruits. Fermented base material may further be obtained from sugarcane juice, palm sap obtained, e.g., from coconut palm, and oil palm, beet root, milk, or a substance of amylaceous (starchy) nature that can be easily converted into simple sugars using enzymes present in cereals or through the addition of suitable malted cereal.

Some embodiments described herein relate to an alcoholic beverage comprising a base alcoholic liquid, one or more neurotransmitter and/or one or more neurotransmitter precursors as defined herein. Such alcoholic beverages are also referred to herein as “enriched alcoholic beverages”. In some embodiments, a contemplated enriched alcoholic beverage optionally further comprises one or more psychostimulant substances as defined herein.

In some embodiments, a contemplated enriched alcoholic beverage comprises at least one dopamine precursor and, optionally, one or more psychostimulant substances as described herein.

Any combination of the base liquid, base alcohol-free liquid and base alcoholic liquid as described herein is contemplated for the enriched alcoholic beverages described herein.

The contemplated enriched alcoholic beverages described herein provide the drinker with the palatability effect of common alcoholic beverages, and further provide the drinker with a sustained euphoric feeling, pleasure and overall sensation of wellbeing and vitality which far exceed the effects provided by corresponding alcoholic beverages which do not contain these enriching supplements and additives. Moreover, unlike common alcoholic beverages, the enriched alcoholic beverages described herein provide a substantial relief of the adverse effects associated with alcohol consumption, particularly the “down” feeling or depression associated with intoxication and sobering up.

A unique and surprising feature of the enriched alcoholic beverage described herein is that they provide the drinker with intensified and prolonged pleasurable, joyful, and euphoric feelings accompanied with a boost of energy and motivation, while substantially reducing drunkenness symptoms.

Contemplated enriched alcoholic beverages provide substantial reduction of intoxication effects, namely, they substantially minimize intoxication effects. For example, these beverages reduce intoxication by 10-95%, or minimize intoxication effects to at least 5% of the effect associated with consuming a corresponding non-enriched alcoholic beverage having the same alcohol content.

In some embodiments, intoxication and/or other negative physical and emotional feelings accompanying sobering up may be relieved, eased or alleviated by about 0 to 100%. For example, alleviation of undesired effects as exerted by a contemplated enriched alcoholic beverage may be up to 10%, up to 20%, up to 30%, up to 40%, up to 50%, up to 60% up to 70%, up to 80%, up to 90%, up to 95%, or up to 100% as compared to a corresponding non-enriched alcoholic beverage. The extent of intoxication relief changes form one person to another and depends on a collective of variables such as the person's tolerance to alcohol, the amount of alcohol in the beverage or the amount of beverage consumed. In some embodiments, the drinker does not experience any insobriety at all even though relatively high amount of alcohol is being consumed, for example, at least 1 liter of enriched beer or at least 3 glasses of enriched wine.

Any combination of neurotransmitter precursors and psychostimulant substances as described herein is contemplated herein.

Because a contemplated enriched alcoholic beverage affords psychotropic effects which outgo the effects of a corresponding non-enriched alcoholic beverage, the enriched beverage may be provided with reduced amounts of alcohol as compared to the corresponding non-enriched alcoholic beverage. For example, the alcohol amount in a contemplated enriched alcoholic beverage may be about 100%, about 95%, about 90%, about 80%, about 75%, about 70%, about 65%, about 60%, about 55%, about 50%, about 45%, about 40%, about 35%, about 30%, about 20%, about 15%, about 10%, about 5%, about 3%, about 1% or about 0.5%, and any individual value therebetween, of the amount of alcohol in a corresponding non-enriched alcoholic beverage.

In some embodiments, the enriched alcoholic beverage is selected from beer, wine, fruit wine, cider, spirit and/or alcopop enriched with a precursor of dopamine, for example, L-tyrosine.

The term “beer” as used herein and in the art means an alcoholic beverage obtained by malting and fermenting one or more of the cereal grains, and includes ale, stout, porter and lager. Typical alcoholic beers include an alcoholic content of 3-8%. Some high-alcohol content beers comprise 8-12% alcohol, for example 10% alcohol.

In some embodiments, the beer is a “reduced alcohol beer”, namely a beer as defined herein comprising up to 3.5% alcohol, for example, up to 3.0%, up to 2.2%, up to 2.0%, up to 1.5%, up to 1.0%, up to 0.5%, up to 0.3%, up to 0.1%, up to 0.05%, up to 0.01%, alcohol, or the beer may be devoid of alcohol.

Non-limiting examples of enriched beers disclosed herein include:

(i) Ale. A type of beer brewed using a warm fermentation method, resulting in a sweet, full-bodied and fruity taste. As with most beers, ale typically can have a bittering agent to balance the sweetness of the malt and act as a preservative. Ale is usually bittered with hops, the dried, cone-shaped flowers of the Mulberry plant. Ale is typically fermented at temperatures between 15 and 24° C. (60 and 75° F., respectively). At temperatures above 24° C. (75° F.) the yeasts can produce significant amounts of esters and other secondary flavour and aroma products, and the result is often a beer with slightly “fruity” compounds resembling those found in fruits such as apple, pear, pineapple, banana, plum, cherry, or prune.

Contemplated enriched Ale beers may be based on, or correspond to known (non-enriched) varieties, for example: (a) Brown Ale, a lightly hopped beer, fairly mildly flavoured, often with a nutty taste. Brown ales range from dark brown beers containing around 3-3.5% alcohol and quite sweet, to red-brown containing 4.5-5% alcohol, and drier beers; (b) Pale Ale, also known as “Bitter Ale”, a beer made from malt dried with coke; (c) India Pale Ale (IPA), a Pale Ale containing extra hops, a beer appreciated for its light and refreshing character; (d) Golden Ale, similar to pale ale but paler, having from 3.5% to 5.3% alcohol; (e) Scotch Ale, a malty, strong ale, amber-to-dark red in color. The malt may be slightly caramelized to impart toffee notes; (f) Barley wine, having from 10% to 12% alcohol, optionally stored for long periods of time, e.g., about 18 to 24 months. Barley wine may taste like massive sweet malt and ripe fruit of pear, orange and lemon, or like darker fruits, chocolate or coffee if darker malts are used; (g) Mild Ale, or unaged ale, having dark brown color and low strength, typically between 3.0 and 3.5% alcohol; (h) Burton Ale, a strong, dark, somewhat sweet ale; (i) Old ale, a malty, medium-strong dark beer (generally above 5% alcohol), may be resemble the traditional English old ales; (j) Belgian Ale, high in alcoholic content but relatively light in body due to the substitution of part of the grist for sucrose, which provides an alcohol boost without adding unfermentable material to the finished product (a process which makes the beer more digestible); and (k) Cask Ale, or cask-conditioned beer, an unfiltered and unpasteurized beer, which is conditioned (including secondary fermentation) and is served from a cask without additional nitrogen or carbon dioxide pressure;

(ii) Porter or Stout. Porter is a dark style of beer made from brown (roasted) malt or roasted barley, hops, water and yeast;

(iii) Lager. Lager (from German: storeroom or warehouse) beer is conditioned at low temperatures. It may be pale, golden, amber, or dark. Lager beer uses a process of cool fermentation, followed by maturation in cold storage. Specific yeast (Saccharomyces pastorianus) is used for brewing lager.

Contemplated enriched lager beers may be based on, or correspond to known (non-enriched) varieties, for example: (a) Pale Lager, such as any one of the common lager beers in worldwide production, for example, Pilsner beer. The flavor of these lighter lagers is usually mild, and it is preferably served refrigerated. Pale lager is a very pale to golden-colored lager with a well attenuated body and noble hop bitterness; (b) Vienna Lager, a reddish-brown or copper-colored beer with medium body and slight malt sweetness. The malt aroma and flavor may have a toasted character; (c) Dark lager, typically ranging in color from amber to dark reddish brown and even black such as a Schwarzbier having a chocolate or liquorice-like flavour similar to stout. Alcohol concentrations of 4.5% to 6% by volume; and (d) Bock, a sweet, relatively strong (6.3%-7.2% alcohol by volume) lager. This beer is clear, ranging in color from light copper to brown, with a bountiful and persistent off-white head; and

(iv) Wheat beer. Wheat beer is a top-fermented beer brewed with a large proportion of wheat relative to the amount of malted barley.

Contemplated enriched wheat beers may be based on, or correspond to known (non-enriched) varieties, for example: (a) a German style beer such as WeiBbier (German—“white beer”), based on the German tradition of mixing at least 50% wheat to barley malt to make a light colored top-fermenting beer; and (b) a Belgian style beer such as Witbier, based on the Belgian tradition of using flavorings such as coriander and orange peel. Belgian style white beers are often made with raw unmalted wheat, as opposed to the malted wheat used in other varieties.

In some embodiments, a disclosed enriched beer comprises one or more dopamine precursors and, optionally, one or more psychostimulant substances as described herein in an amount that imparts to the beer palatability and/or positive psychotropic effects as defined herein and/or positive psychoactive effects as described herein, and/or pleasure of drinking, which are at least as those provided by a corresponding non-enriched and/or any known beer containing 3.8-10% alcohol by volume. In some embodiments, a contemplated enriched beer provides positive psychoactive effects such as euphoric and joyful feeling, which exceed the effects exerted by a corresponding non-enriched beer containing 3.8-10% alcohol by volume, while substantially minimizing intoxication effects.

In the enriched beer described herein, the amount of dopamine precursor, for example, tyrosine, is within a range of from about 0.10 mg/ml to 5.0 mg/ml, for example, from about 0.10 mg/ml to about 0.20 mg/ml, from about 0.10 mg/ml to about 0.30 mg/ml, from about 0.25 mg/ml to about 0.45 mg/ml, from about 0.30 mg/ml to about 0.50 mg/ml, from about 0.30 mg/ml to about 0.60 mg/ml, from about 0.45 mg/ml to about 0.65 mg/ml, from about 0.50 mg/ml to about 0.70 mg/ml, from about 0.50 mg/ml to about 0.80 mg/ml, from about 0.60 mg/ml to about 0.90 mg/ml, from about 0.80 mg/ml to about 1.00 mg/ml, from about 0.90 mg/ml to about 1.10 mg/ml, from about 0.95 mg/ml to about 1.25 mg/ml, from about 1.00 mg/ml to about 1.30 mg/ml, from about 1.20 mg/ml to about 1.50 mg/ml, from about 1.45 mg/ml to about 1.75 mg/ml, from about 1.75 mg/ml to about 2.10 mg/ml, from about 2.00 mg/ml to about 2.50 mg/ml, from about 2.45 mg/ml to about 3.00 mg/ml, from about 2.50 mg/ml to about 3.50 mg/ml, from about 3.00 mg/ml to about 4.00 mg/ml, from about 3.75 mg/ml to about 4.50 mg/ml, or from about 4.35 mg/ml to about 5.00 mg/ml, including any subranges and any intermediate values therebetween.

In exemplary embodiment, a disclosed enriched beer comprises from about 0.1 mg/ml to about 0.45 mg/ml of tyrosine.

When a contemplated enriched beer comprises a psychostimulant substance, the amount of the psychostimulant substance, for example, caffeine, may be within a range of from 0.01 mg/ml to 0.04 mg/ml, 0.02 mg/ml to 0.05 mg/ml, 0.05 mg/ml to 0.08 mg/ml, 0.05 to 0.09 mg/ml, 0.05 to 0.10 mg/ml, 0.08 to 0.10 mg/ml, 0.08 to 0.12 mg/ml, 0.09 to 0.15 mg/ml, 0.10 to 0.15 mg/ml, 0.12 to 0.18 mg/ml, 0.12 to 0.20 mg/ml, 0.15 to 0.25 mg/ml, 0.20 to 0.25 mg/ml, 0.25 to 0.45 mg/ml, 0.25 to 0.50 mg/ml, 0.45 to 0.60 mg/ml, 0.50 to 0.75 mg/ml, 0.65 to 0.85 mg/ml, 0.85 to 0.95 mg/ml, 0.95 to 1.00 mg/ml, 0.95 to 1.30 mg/ml, 1.00 to 1.30 mg/ml, 1.00 to 1.50 mg/ml, 1.25 to 1.75 mg/ml, 1.50 to 2.00 mg/ml, 2.00 to 3.00 mg/ml, 3.00 to 5.00 mg/ml, 3.50 to 5.00 mg/ml, 4.50 to 6.00 mg/ml, or 6.00 to 9.00 mg/ml, including any subranges and any intermediate values therebetween.

In some embodiment, a disclosed enriched beer comprises from about 0.01 mg/ml to about 0.9 mg/ml (or 10 mg/L to about 900 mg/L) caffeine.

An exemplary enriched beer described herein is a lager beer comprising from about 0.1 mg/ml to about 0.6 mg/ml of tyrosine and from about 0.01 mg/ml to about 0.75 mg/ml caffeine.

In some embodiments, the base alcoholic beverage of an enriched alcoholic beverage described herein is wine. The wine may have an alcohol content of 10-14% by volume. The term “wine” as used herein refers to the fermented juice of grapes, made in many varieties, such as red, white, sweet, dry, still, and sparkling. Exemplary wine beverages include, but are not limited to, dry red or white wine; semi-dray red or white wine; rosé wine; dessert wine such as Muscato wine; fortified wine such as Marsala, Port, Madeira, Sherry, vinsanto, and vermouth; and sparkling wine such as Champagne.

In some embodiments, an enriched wine is a “reduced alcohol wine”, namely a wine as defined herein comprising up to 8% alcohol, for example, up to 7%, up to 6%, up to 5%, up to 4.5%, up to 4%, up to 3.5%, up to 3%, up to 1%, up to 0.5%, up to 0.1%, up to 0.05%, or the wine may be devoid of alcohol.

In some embodiments, an enriched wine is contemplated, comprising a dopamine precursor and, optionally, a psychostimulant substance as described herein in an amount that imparts to the wine palatability and/or positive psychotropic effects and/or positive psychoactive effects as defined herein, and/or pleasure of drinking, at least as those provided by a corresponding non-enriched wine containing.

In certain embodiments, the enriched wine contemplated herein affords positive psychoactive effects such as euphoric and joyful feeling, which exceed the effects exerted by a corresponding non-enriched wine containing 12-14% alcohol by volume, while substantially minimizing intoxication effects.

In a contemplated enriched wine, the amount of dopamine precursor, for example, tyrosine, may be within a range of from about 0.10 mg/ml to 5.0 mg/ml, for example, from about 0.10 to about 0.30 mg/ml, from about 0.25 to about 0.45 mg/ml, from about 0.30 to about 0.50 mg/ml, from about 0.30 to about 0.60 mg/ml, from about 0.35 to about 0.65 mg/ml, from about 0.45 to about 0.65 mg/ml, from about 0.50 to about 0.70 mg/ml, from about 0.50 to about 0.80 mg/ml, from about 0.60 to about 0.90 mg/ml, from about 0.80 to about 0.95 mg/ml, from about 0.80 to about 1.00 mg/ml, from about 0.90 to about 1.10 mg/ml, from about 0.95 to about 1.25 mg/ml, from about 1.00 to about 1.30 mg/ml, from about 1.10 to about 1.50 mg/ml, from about 1.50 to about 1.80 mg/ml, from about 1.55 to about 1.95 mg/ml, from about 1.70 to about 2.00 mg/ml, from about 1.95 to about 2.50 mg/ml, from about 2.55 to about 2.95 mg/ml, from about 2.80 to about 3.50 mg/ml, from about 3.50 to about 4.00 mg/ml, from about 3.80 to about 4.50 mg/ml, or from about 4.50 to about 5.00 mg/ml, including any subranges and any individual values therebetween.

In certain embodiment, an enriched wine contemplated herein comprises from about 0.1 mg/ml to about 1.80 mg/ml of tyrosine.

When the enriched wine described herein comprises a psychostimulant substance, for example, caffeine, its amount may be within a range of from 0.01 mg/ml to 0.04 mg/ml, 0.02 to 0.05 mg/ml, 0.05 to 0.10 mg/ml, 0.08 to 0.10 mg/ml, 0.08 to 0.12 mg/ml, 0.09 to 0.15 mg/ml, 0.10 to 0.15 mg/ml, 0.12 to 0.18 mg/ml, 0.12 to 0.20 mg/ml, 0.15 to 0.25 mg/ml, 0.20 to 0.25 mg/ml, 0.25 to 0.45 mg/ml, 0.25 to 0.50 mg/ml, 0.45 to 0.60 mg/ml, 0.50 to 0.75 mg/ml, 0.65 to 0.85 mg/ml, 0.85 to 0.95 mg/ml, 0.95 to 1.00 mg/ml, 0.95 to 1.30 mg/ml, 1.00 to 1.35 mg/ml, 1.00 to 1.50 mg/ml, 1.25 to 1.75 mg/ml, or 1.50 to 2.00 mg/ml, including any subranges and individual values therebetween.

In exemplary embodiments, the enriched wine contemplated herein comprises from about 0.01 mg/ml to about 0.75 mg/ml (or 10 mg/L to about 750 mg/L) caffeine.

The term “spirit” as used herein and in the art refers to a distilled alcohol beverage obtained, for example, by distilling starchy material, and includes, without being limited to, variety of raw grain alcohols, brandies, liquors, saki, Ouzo, arrack, rum, vodka, tequila, schnapps, whiskey, gin, cordial, Cachaca, absinthe, baijiu, eau de vie, soju, aguardiente, palinka, fernet, slivovitz and the like.

In some embodiments, a contemplated enriched spirit is a “reduced alcohol spirit”, namely a spirit as defined herein comprising up to 50% alcohol, for example, up to 45%, up to 40%, up to 35%, up to 30%, up to 25%, up to 20%, up to 15%, up to 10%, up to 8%, up to 7%, up to 6%, up to 5%, up to 4%, up to 3.5%, up to 3%, up to 2%, up to 1%, up to 0.5% alcohol by volume, or the spirit may be devoid of alcohol.

In some embodiments, an enriched spirit is contemplated, comprising one or more dopamine precursors and, optionally, one or more psychostimulant supplements as described herein in an amount that imparts to the spirit palatability and/or positive psychotropic effects and/or positive psychoactive effects as described herein, and/or pleasure of drinking, at least as those provided by any known corresponding non-enriched spirit. In certain embodiments, an enriched spirit contemplated herein affords positive psychoactive effects such as euphoric and joyful feeling, which exceed the effects exerted by a corresponding non-enriched spirit, while substantially minimizing intoxication effects.

In the enriched spirit described herein, the amount of dopamine precursor, for example, tyrosine, may be within a range of from about 0.10 mg/ml to 5.0 mg/ml, for example, from about 0.10 to about 0.30 mg/ml, from about 0.25 to about 0.45 mg/ml, from about 0.30 to about 0.50 mg/ml, from about 0.30 to about 0.60 mg/ml, from about 0.35 to about 0.65 mg/ml, from about 0.45 to about 0.65 mg/ml, from about 0.50 to about 0.70 mg/ml, from about 0.50 to about 0.80 mg/ml, from about 0.60 to about 0.90 mg/ml, from about 0.80 to about 0.95 mg/ml, from about 0.80 to about 1.00 mg/ml, from about 0.90 to about 1.10 mg/ml, from about 0.95 to about 1.25 mg/ml, from about 1.00 to about 1.30 mg/ml, from about 1.10 to about 1.50 mg/ml, from about 1.50 to about 1.80 mg/ml, from about 1.55 to about 1.95 mg/ml, from about 1.70 to about 2.00 mg/ml, from about 1.95 to about 2.50 mg/ml, from about 2.55 to about 2.95 mg/ml, from about 2.80 to about 3.50 mg/ml, from about 3.50 to about 4.00 mg/ml, from about 3.80 to about 4.50 mg/ml, or from about 4.50 to about 5.00 mg/ml, including any subranges and individual values therebetween.

In exemplary embodiments, an enriched spirit contemplated herein comprises from about 0.1 mg/ml to about 2.5 mg/ml of tyrosine.

When an enriched spirit described herein comprises a psychostimulant supplement, the amount of psychostimulant supplement, for example, caffeine, may be within a range of from 0.01 mg/ml to 0.04 mg/ml, 0.02 to 0.05 mg/ml, 0.05 to 0.10 mg/ml, 0.08 to 0.10 mg/ml, 0.08 to 0.12 mg/ml, 0.09 to 0.15 mg/ml, 0.10 to 0.15 mg/ml 0.10 to 0.17 mg/ml, 0.12 to 0.18 mg/ml, 0.12 to 0.20 mg/ml, 0.15 to 0.25 mg/ml, 0.20 to 0.25 mg/ml, 0.23 to 0.35 mg/ml, 0.25 to 0.45 mg/ml, 0.25 to 0.50 mg/ml, 0.45 to 0.60 mg/ml, 0.50 to 0.75 mg/ml, 0.65 to 0.85 mg/ml, 0.70 to 0.95 mg/ml, 0.95 to 1.00 mg/ml, 0.95 to 1.30 mg/ml, 1.00 to 1.30 mg/ml, 1.00 to 1.50 mg/ml, 1.25 to 1.75 mg/ml, 1.50 to 2.00 mg/ml, 1.60 to 2.30 mg/ml, or 2.30 to 2.60 mg/ml, including any subranges and any individual values therebetween.

In exemplary embodiments, the enriched spirit contemplated herein comprises from about 0.01 mg/ml to about 0.75 mg/ml (or 10 mg/L to about 750 mg/L) caffeine.

Other known alcoholic beverages which can be made enriched alcoholic beverages in accordance with embodiments described herein, include, but are not limited to, Desi dam, made by fermenting molasses or high sugar containing fruits; Huangjiu (Chinese, made from rice, millet, or wheat using a special starter culture of yeast, mold, and bacteria); Icariine Liquor; Kasiri (made from cassava); Kilju (Finnish, made from sugar); Kumis (central asia, traditionally made from horse milk but now primarily cow milk); Mead (made from honey); Nihamanchi (South America) a.k.a. nijimanche (Ecuador and Peru) (made from cassava); Palm wine (made from the sap of various palm trees); Parakari (made from cassava); Pulque (originally made by the natives of Mexico, made from the sap of the maguey plant); Sakura (made from cassava); Sake (made from rice); Sonti; Tepache; Tiswin (made from corn or saguaro, a large cactus); and Tonto.

Alcopop beverages, also termed herein and in the art “coolers” or “spirit coolers”, are flavored alcoholic beverages or flavored malt beverages based on fruit juice or nectar, and/or a variety of naturally and/or artificially flavored syrups. Exemplary alcopop beverages include, but are not limited to: (i) a malt beverage, designate herein “a beer cooler”, containing a malt base or beer and at least 5% by volume of added natural or artificial blending material, such as fruit juice, flavorants, coloring agents, and, optionally, preservatives; (ii) a wine cooler which is a beverage containing grape wine and more than 15% by volume of added natural or artificial blending material, such as fruit juices, flavorants, adjuncts, water (plain, carbonated, or sparkling), coloring agents, and, optionally, preservatives; and (iii) a beverage designated herein “a spirit cooler”, containing distilled alcohol, and added natural or artificial blending material, such as fruit juices, flavorants, coloring agents, and, optionally, preservatives.

Alcopop brands are numerous, and their alcoholic base vary greatly. Most alcopop beverages contain 3.8-7% alcohol by volume, and some may even contain as much as 12.5% alcohol by volume. Some notable brands include, but are not limited to, Smirnoff Ice, Mike's Hard Lemonade, Bacardi Breezer, Skyy Blue, Jack Daniel's Hard Cola, WKD Original Vodka, Six Degrees and MG Spirits.

In some embodiments, an enriched cider and/or enriched alcopop beverage is contemplated, comprising one or more dopamine precursors and, optionally, one or more psychostimulant supplements as described herein in an amount that imparts to the cider or alcopop palatability and/or positive psychotropic effects and/or positive psychoactive effects as described herein, and/or pleasure of drinking, at least as those provided by a corresponding non-enriched or any known alcopop or cider.

In some embodiments, the enriched alcopop or enriched cider contemplated herein affords positive psychoactive effects such as euphoric and joyful feeling, which exceed the effects exerted by a corresponding non-enriched alcopop or non-enriched cider containing 3.8-5.0% alcohol by volume, while substantially minimizing intoxication effects.

In an enriched alcopop described herein, the amount of dopamine precursor, for example, tyrosine, is within a range of from about 0.10 mg/ml to 5.0 mg/ml, for example, from about 0.10 mg/ml to about 0.20 mg/ml, from about 0.10 to about 0.30 mg/ml, from about 0.25 to about 0.45 mg/ml, from about 0.30 to about 0.50 mg/ml, from about 0.30 to about 0.60 mg/ml, from about 0.35 to about 0.65 mg/ml, from about 0.45 to about 0.65 mg/ml, from about 0.45 to about 0.70 mg/ml, from about 0.50 to about 0.70 mg/ml, from about 0.50 to about 0.80 mg/ml, from about 0.60 to about 0.90 mg/ml, from about 0.80 to about 0.95 mg/ml, from about 0.80 to about 1.00 mg/ml, from about 0.90 to about 1.10 mg/ml, from about 0.95 to about 1.25 mg/ml, from about 1.00 to about 1.30 mg/ml, from about 1.10 to about 1.30 mg/ml, from about 1.20 to about 1.50 mg/ml, from about 1.55 to about 1.75 mg/ml, from about 1.75 to about 2.10 mg/ml, from about 1.95 to about 2.50 mg/ml, from about 2.55 to about 2.95 mg/ml, from about 2.80 to about 3.50 mg/ml, from about 3.50 to about 4.00 mg/ml, from about 3.80 to about 4.50 mg/ml, or from about 4.50 to about 5.00 mg/ml including any subranges and any individual values therebetween.

In exemplary embodiment, an enriched alcopop contemplated herein comprises from about 0.1 mg/ml to about 0.45 mg/ml of tyrosine.

When an enriched alcopop described herein comprises a psychostimulant supplement, the amount of psychostimulant supplement, for example, caffeine, may be within a range of from 0.01 mg/ml to 0.04 mg/ml, 0.02 to 0.05 mg/ml, 0.05 to 0.10 mg/ml, 0.08 to 0.10 mg/ml, 0.08 to 0.12 mg/ml, 0.09 to 0.15 mg/ml, 0.10 to 0.15 mg/ml, 0.12 to 0.18 mg/ml, 0.12 to 0.20 mg/ml, 0.15 to 0.25 mg/ml, 0.20 to 0.25 mg/ml, 0.25 to 0.45 mg/ml, 0.25 to 0.50 mg/ml, 0.45 to 0.60 mg/ml, 0.50 to 0.75 mg/ml, 0.65 to 0.85 mg/ml, 0.85 to 0.95 mg/ml, 0.95 to 1.00 mg/ml, 0.95 to 1.30 mg/ml, 1.00 to 1.30 mg/ml, 1.00 to 1.50 mg/ml, 1.25 to 1.75 mg/ml, or 1.50 to 2.00 mg/ml, including any subranges and any individual values therebetween.

In exemplary embodiments, an enriched alcopop contemplated herein comprises from about 0.01 mg/ml to about 0.75 mg/ml (or 10 mg/L to about 750 mg/L) caffeine.

In some embodiments, the enriched alcoholic beverage is a fruit wine. In some embodiments, a contemplated fruit wine comprises fermented base material produced from one or more natural or artificially flavored fruit juice such as, but not limited to, grapefruit, orange, apple, berries, lemon, peach, apricot, plum, pomegranate, fig, dates, mango, melon, water melon, pear, guava, pineapple, passion fruit, kiwi, banana, litchi, papaya, and any combination thereof. Herein, “fruit wine” does not include grape wine.

In some embodiments, the fermented base material is apple wine.

Apple wine and apple cider are not to be confused as referring to the same alcoholic beverage herein. The term “apple cider”, as used herein, refers to apple juice that has been allowed to ferment. The natural sugars in the apple juice ferment to an alcohol level that runs somewhere around 3% to 6%. “Apple wine” is also a fermented apple juice but it is much higher in alcohol than cider. It is essentially apple juice that has gone through the same process for making wine. The acidity is adjusted, nutrients are used, and, it is bottled and aged. But, most significant of all, sugar is added to the fermentation to bring up the final alcohol level of the apple wine. Most apple wines are around 10% to 12% alcohol by volume.

There is also a difference between how apple wine and apple cider keep and age. Cider needs to be consumed more quickly than apple wine. The lower level of alcohol makes it less stable. The use of preservatives and maybe even constant refrigeration will help in this regard, but still apple cider is a drink that is to be consumed in weeks and months, not years. Apple wine, on the other hand, is more stable. Its higher alcohol content makes it less susceptible to spoilage and general flavor deterioration. Like most wines, it will taste a little harsh at bottle time. It needs time to age out its harshness. Typically, an apple wine will be very drinkable at around 3 months of aging and at its best at around 9 months.

Also, apple cider and apple wine taste differently; while cider tastes a lot like apple juice, apple wine barely maintains an apple flavor. As such, any flavorants added to apple wine bestow to it their flavors. For example, supplementing apple wine with extracts of mint, grapefruit, orange, pineapple or lime will produce mint, grapefruit, orange, pineapple, or lime-flavored apple wine.

In an enriched fruit wine described herein, e.g., enriched apple wine, the amount of dopamine precursor, for example, tyrosine, is within a range of from about 0.10 mg/ml to 10.0 mg/ml, for example, from about 0.10 mg/ml to about 0.20 mg/ml, from about 0.10 to about 0.30 mg/ml, from about 0.25 to about 0.45 mg/ml, from about 0.30 to about 0.50 mg/ml, from about 0.30 to about 0.60 mg/ml, from about 0.35 to about 0.65 mg/ml, from about 0.45 to about 0.65 mg/ml, from about 0.45 to about 0.70 mg/ml, from about 0.50 to about 0.70 mg/ml, from about 0.50 to about 0.80 mg/ml, from about 0.80 to about 1.00 mg/ml, from about 0.90 to about 1.10 mg/ml, from about 0.95 to about 1.25 mg/ml, from about 1.00 to about 1.30 mg/ml, from about 1.10 to about 1.30 mg/ml, from about 1.20 to about 1.50 mg/ml, from about 1.55 to about 1.75 mg/ml, from about 1.75 to about 2.10 mg/ml, from about 1.95 to about 2.50 mg/ml, from about 2.55 to about 2.95 mg/ml, from about 2.80 to about 3.50 mg/ml, from about 3.50 to about 4.00 mg/ml, from about 3.80 to about 4.50 mg/ml, from about 4.50 to about 5.00 mg/ml, from about 4.75 to about 6.00 mg/ml, from about 5.20 to about 6.50 mg/ml, from about 6.25 to about 7.50 mg/ml, from about 7.00 to about 8.00 mg/ml, from about 7.75 to about 9.00 mg/ml, from about 8.50 to about 9.50 mg/ml, or from about 9.00 to about 10.00 mg/ml, including any subranges and any individual values therebetween.

In exemplary embodiments, an enriched fruit wine contemplated herein comprises about 100 mg/L, about 200 mg/L, about 300 mg/L, about 400 mg/L, or about 500 mg/L, about 750 mg/ml or from about 100 mg/L to about 1000 mg/liter tyrosine.

When a fruit wine described herein comprises a psychostimulant supplement, the amount of psychostimulant supplement, for example, caffeine, may be within a range of 0.01 mg/ml to 0.04 mg/ml, 0.02 to 0.05 mg/ml, 0.05 to 0.10 mg/ml, 0.08 to 0.10 mg/ml, 0.08 to 0.12 mg/ml, 0.09 to 0.15 mg/ml, 0.10 to 0.15 mg/ml, 0.12 to 0.18 mg/ml, 0.12 to 0.20 mg/ml, 0.15 to 0.25 mg/ml, 0.20 to 0.25 mg/ml, 0.25 to 0.45 mg/ml, 0.25 to 0.50 mg/ml, 0.45 to 0.60 mg/ml, 0.50 to 0.75 mg/ml, 0.65 to 0.85 mg/ml, 0.85 to 0.95 mg/ml, 0.95 to 1.00 mg/ml, 0.95 to 1.30 mg/ml, 1.00 to 1.30 mg/ml, 1.00 to 1.50 mg/ml, 1.25 to 1.75 mg/ml, or 1.50 to 2.00 mg/ml, including any subranges and any individual values therebetween.

In exemplary embodiments, an enriched fruit wine contemplated herein e.g, apple wine, comprises from about 0.01 mg/ml to about 2.00 mg/ml (or 10 mg/L to about 200 mg/L) about 100 mg/L, about 150 mg/L, or about 350 mg/L caffeine.

The alcohol in the enriched fruit wine, at least in some embodiments, may be obtained solely from fermentation of sugars in the fermentable base material, with no addition of distilled alcohol, i.e., the beverage is not distilled.

In some embodiments, an enriched fruit wine beverage is contemplated, comprising one or more dopamine precursors and, optionally, one or more psychostimulant supplements as described herein in an amount that imparts to the beverage palatability and/or positive psychotropic effects and/or positive psychoactive effects as described herein, and/or pleasure of drinking, at least as those provided by a corresponding non-enriched or any known alcopop or cider.

In some embodiments, the enriched fruit wine contemplated herein affords positive psychoactive effects such as euphoric and joyful feeling, which exceed the effects exerted by a corresponding non-enriched fruit wine containing 10-12% alcohol by volume, while substantially minimizing intoxication effects.

Herein throughout, whenever a percentage (%) is indicated, % volume of the total volume (% v/v) of the beverage is meant, unless otherwise indicated.

In some of any one of the embodiments of the present disclosure, optionally or additionally, the base material of the a contemplated alcoholic food product, for example, base liquid or base alcoholic liquid, is formulated to include additives, such as sugar or alternative natural and/or artificial sweeteners, flavoring agents, colorants, odoriferous agents, enzymes, CO₂ and/or other additives such as viscosity modifying agents, foaming agents, antifoaming agents, and preservatives that account for the taste and texture of, e.g., wine or beer or spirit, such that the enriched alcoholic product will contain at least part or all the ingredients used to form the corresponding non-enriched alcoholic product. Preferably, additives used in the enriched alcohol food products described herein are FDA-approved, and/or edible. In some embodiments, the additives are selected as soluble in the base material, e.g., base alcoholic beverage or base liquid.

In some embodiments, a contemplated enriched alcoholic beverage disclosed herein may comprise relatively low sugar content. Herein, a low sugar content is defined as overall sugar concentration of up to 140 gr/L. Exemplary ranges and subranges of total sugar concentrations include, e.g., from about 0 gr/L to about 5 gr/L, from about 3 gr/L to about 8 gr/L, from about 5 gr/L to about 10 gr/L, from about 8 gr/L to about 15 gr/L, from about 10 gr/L to about 20 gr/L, from about 15 gr/L to about 25 gr/L, from about 20 gr/L to about 35 gr/L, from about 30 gr/L to about 55 gr/L, from about 35 gr/L to about 45 gr/L, from about 40 gr/L to about 45 gr/L, from about 55 gr/L to about 65 gr/L, from about 50 gr/L to about 70 gr/L, from about 60 gr/L to about 75 gr/L, from about 70 gr/L to about 85 gr/L, from about 80 gr/L to about 100 gr/L, from about 90 gr/L to about 120 gr/L, and from about 100 gr/L to about 400 gr/L.

In some embodiments, a contemplated alcoholic beverage contains up to 70 mg/L sugar in total. Non-limiting examples of such contemplated beverages include beverages comprising sugar in total amount of from about 20 gr/L to about 40 gr/L, from about 25 gr/L to about 70 gr/L, from about 30 gr/L to about 60 gr/L, from about 40 gr/L to about 55 gr/L, about 45 gr/L or about 50 gr/L. In exemplary embodiments, the low sugar alcoholic beverage is an enriched fruit wine, for example, enriched apple wine.

The terms “flavoring agent” and “odoriferous agent”, as used herein, describe a class of substances which are added to edible products in order to induce a certain flavor or smell in the product, respectively, and are commonly referred to herein as “flavorants” and “taste-improving additives”. The flavorants can be synthetic or natural extracts extracted from a source substance. Typical flavorants are specific and often complex mixtures of singular naturally occurring or synthetic flavor compounds combined together to either imitate or enhance a natural flavor. Many flavorants are esters, which can be characterized by a typical flavor, such as diacetyl which gives a buttery flavor, isoamyl acetate that is perceived as banana, cinnamic aldehyde which is the basis for the typical flavor of cinnamon, ethyl propionate which is perceived as fruity, limonene that is perceived as orange, ethyl-(E, Z)-2,4-decadienoate that is perceived as pear, allyl hexanoate that is perceived as pineapple, ethyl maltol, which is perceived as sugar or cotton candy, methyl salicylate that is known as the wintergreen flavor, and benzaldehyde that is perceived as bitter almond. Further synthetic flavorant are exemplified by amaretto, cola and ice cream flavors

Flavoring agent of a natural source can be, for example, an extract, oil or juice of a fruit, vegetable, herb or of any other edible substance, and any combination thereof. Extracts, juice and/or oil serving as natural flavorants may be obtained, for example, form fruits and herbs such as, but not limited to, grapefruit, mint, grapes, orange, elder, ginger, apple, anise, lemon grass, a berry such as blueberry, strawberry, cranberry and the like, peach, apricot, plum, pomegranate, fig, dates, mango, melon, water melon, pear, guava, pineapple, passion fruit, kiwi, banana, litchi, papaya, and any combination thereof.

Any one or more of these flavorant may optionally be added to a contemplated beverage.

The terms “colorant” and “coloring agent”, as used herein, are interchangeably referring to any natural or synthetic coloring substance that is added to food or drink in order to alter its color. Exemplary usable colorants include, but are not limited to, synthetic colorants such as FD&C Blue No. 1—Brilliant Blue FCF (E133), FD&C Blue No. 2—Indigotine (E132), FD&C Green No. 3—Fast Green FCF (E143), FD&C Red No. 40—Allura Red AC (E129), FD&C Red No. 3—erythrosine (E127), FD&C Yellow No. 5—tartrazine (E102), and FD&C Yellow No. 6—Sunset Yellow FCF (E110), and natural food colorants such as carmine (E120), enocianin (E163), black carrot (E163), paprika (E160c), annatto (E160b), beta carotene (E160a), lutein (E161b), riboflavin (E101), curcumin (E100), copper chlorophyllin (E141), chlorophyll (E140), caramel (E150), and extracts of foodstuffs such as elderberry, aronia, grape, beetroot, carrot, turmeric (tumeric) root, spinach, stinging nettle and burnt sugar (caramelized sugar).

The term “preservative”, as used herein, describes a synthetic or natural additive substance that is added to edible products in order to prevent or retard chemical and biochemical decomposition of the product by oxygen, moisture and/or microbes. Exemplary anti-microbial preservatives include, but are not limited to, calcium propionate, sodium nitrate, sodium nitrite, sulfites (sulfur dioxide, sodium bisulfite, potassium hydrogen sulfite, etc.), disodium EDTA, sodium benzoate, potassium sorbate. Natural substances that retard microorganisms growth include lactic acid, salt, sugar and vinegar.

Exemplary antioxidant preservatives include, but are not limited to, butylated hydroxyanisole (BHA) and butylated hydroxytoluene (BHT). Natural antioxidants include, but are not limited to, herbal extracts such as rosemary and oregano, and vitamins such as Vitamin E and Vitamin C (ascorbic acid).

The term “foaming agent”, as used herein, describes an edible surfactant, which when present in small amounts, facilitates the formation of a foam, or enhances its colloidal stability by inhibiting the coalescence of bubbles. Exemplary foaming agents include, without limitation, sodium lauryl sulfate (SLS), sodium lauryl ether sulfate (SLES) and ammonium lauryl sulfate (ALS).

The term “antifoaming agent”, as used herein, describes an edible substance that inhibits the formation of foam and curbs effusion or effervescence in edible products. An exemplary antifoaming agent is polydimethylsiloxane.

The terms “viscosity modifying agent” or “thickener”, as used herein, are interchangeable and describe agents that enable to control the viscosity of an enriched alcoholic food products described herein. Exemplary thickeners include, but are not limited to, starch-based thickeners such as maltodextrin and gum-based thickeners such as xanthan or cellulose gum.

A disclosed enriched alcoholic beverage, in accordance with some embodiments, may comprise fruit wine, from about 3% to about 21% alcohol by volume, up to about 140 gr/L sugar, flavorants, at least one neurotransmitter precursor and, optionally at least one psychostimulant substance. The alcohol in the beverage, at least in some embodiments, is obtained solely from fermentation of sugars in the fermentable base material, with no addition of distilled alcohol, wherein the fermentable base material is at least one of a natural or artificially flavored fruit juice, berry juice, vegetable juice, fruit syrup, concentrate or nectar from fruits.

In exemplary embodiments, an alcoholic beverage is contemplated, comprising from about 85% to about 95% apple wine by volume, from about 8% to about 11% undistilled alcohol by volume, from about 45 gr/L to about 55 gr/L sugar, from about 350 mg/L to about 450 mg/L tyrosine, from about 140 mg/L to about 150 mg/L caffeine, and a flavorant having a flavor selected from bitter lime, lemon, grapefruit, mint, grape, orange, ginger, apple, anise, a berry, lemon grass, peach, apricot, plum, pomegranate, fig, dates, mango, melon, water melon, pear, guava, pineapple, passion fruit, kiwi, banana, litchi, papaya, and any combination thereof.

An enriched alcoholic product described herein may be contained in any of the known containers applied for alcoholic products in general. For example, when the enriched alcoholic product is a beverage, for example a beer, it may be contained and marketed in a 330 ml, 500 ml, 750 ml and 1-liter bottle or tin, or in a barrel. The stability of a contemplated enriched alcoholic food product is preferably more than 365 days at room temperature, and it may be stored for up to 999 days at 10° C.

In a further aspect, the present disclosure relates to a process for the preparation of an enriched alcoholic beverage. A contemplated process comprises at least the steps of mixing a base liquid as defined herein with an amount of one or more neurotransmitter precursors as described herein. In some embodiments, the process further comprises the addition of one or more psychostimulant supplements as described herein. In some embodiments, the base liquid is an alcoholic beverage such as beer, wine, fruit wine, spirit, cider or alcopop.

In some embodiments, the base liquid is a non-alcoholic beverage (e.g., juice, water and the like), and one or more neurotransmitter precursors and, optionally one or more psychostimulant supplements may be added to the non-alcoholic base beverage together with a desired amount of alcohol.

In some embodiments, the enriched alcoholic beverage, for example beer, is prepared by brewing the beer together with neurotransmitter precursor(s) and the psychostimulant supplement(s). Likewise, wine and spirit may be fermented or distilled, respectively, in processes that are adjusted to includes steps of addition of neurotransmitter precursor(s) and psychostimulant supplement(s), as long as the fermentation, distillation or brewing processes maintain the neurotransmitter precursor(s) and the psychostimulant supplement(s) intact and bioavailable, and further provided that the palatability, texture and appearance of the beverage in not substantially affected by including neurotransmitter precursor(s) and/or the psychostimulant supplement(s) in the process of preparation thereof.

In some embodiments, the process described herein is for the preparation of enriched beers comprising one or more dopamine precursors and, optionally further comprising one or more psychostimulant supplements. In some exemplary embodiments, a contemplated process comprises the addition of tyrosine and caffeine to a base alcoholic beer. In alternative embodiments, an enriched beer is brewed together with tyrosine and caffeine in the brewery.

### Methods of Enhancing Euphoric Sensation and Improving Cognitive Abilities

The present disclosure is based on the discovery by the present inventor that some emotional and mental situations, as well as some cognitive abilities, mediated by release of certain neurotransmitters in the central nervous system (CNS) and boosted by alcohol consumption, may be strengthened, enhanced or benefit if one or more of these neurotransmitters or precursors thereof are provided to a subject when alcohol is consumed. Hence it has been envisaged by the present inventor that cognitive abilities such as concentration, focus, alertness, motivation, learning, creativity, curiosity, pursuing rewarding feelings of satisfaction and/or pleasure as well as feelings or emotions such as joy, euphoria and the like, which are mediated by the same neurotransmitters that are affected by, or related to, alcohol consumption, may be manipulated, for example, enhanced or improved. Moreover, it has been envisaged by the present inventor that the effects associated with alcohol consumption, for example, via drinking alcoholic beverages, particularly the pleasurable effects, may be enhanced and prolonged by consuming alcohol together with at least one neurotransmitter or a precursor thereof, which is directly or indirectly associated with positive psychotropic affects, even without increasing the amount of alcohol in the product. It has been further envisaged by the present inventor that certain desired cognitive effects or qualities associated with alcohol consumption may be improved by combining the consumption of both alcohol and at least one such neurotransmitter or precursor thereof.

For example, disclosed herein is a discovery that the euphoric perception or feeling accompanying alcohol consumption may be enhanced and prolonged when alcohol is consumed together with a precursor of dopamine, optionally with further consumption of a psychoactive stimulant like caffeine. This euphoric feeling lasts longer than that induced by consuming alcohol alone and, furthermore, it is not accompanied by undesired physiological and psychological short-term effects associated with intoxication such as gastric irritation, numbing, stupor or daze, and mood swings.

In one aspect, the present disclosure relates to a method for enhancing and prolonging in a subject a euphoric sensation associated with alcohol consumption, the method comprising providing to the subject, concomitantly upon alcohol consumption, an effective amount of at least one neurotransmitter or a precursor thereof, thereby enhancing and prolonging a euphoric sensation in the subject.

As used herein, the terms “euphoria”, “euphoric sensation”, “euphoric feeling”, and “a high feeling” are interchangeable and refer to the experience (or affect) of pleasure or excitement, and intense feelings of well-being and happiness.

In a further aspect, the present disclosure relates to a method for boosting or improving a cognitive ability in a subject, the method comprising providing to the subject an effective amount of at least one neurotransmitter or a precursor thereof and alcohol, thereby boosting or improving a cognitive ability in the subject. In some embodiments, the at least one neurotransmitter or a precursor thereof are provided to a subject concomitantly upon alcohol consumption by the subject.

In yet a further aspect, the present disclosure relates to a method for boosting or enhancing a mental process in a subject, the method comprising providing to the subject an effective amount of at least one neurotransmitter or a precursor thereof and alcohol, thereby boosting or enhancing a mental process in the subject. In some embodiments, the at least one neurotransmitter or a precursor thereof are provided to a subject concomitantly upon alcohol consumption by the subject.

In some embodiments, the enhanced euphoric sensation, the improved cognitive ability and/or the enhanced mental process, in accordance with a disclosed method, lasts, for example, for up to 24 or 48 hours after consumption of alcohol and at least one neurotransmitter or a precursor thereof.

Cognition refers herein to a conscious mental activity such as thinking, reasoning, understanding, learning, and remembering. Cognition has to do with how a person understands the world and acts in it. “Cognitive abilities” or “cognitive skills”, interchangeably used herein, are foundational brain-based skills needed to carry out any task from the simplest to the most complex. They relate not to actual knowledge but rather to the mechanisms of learning, remembering, problem-solving, and paying attention. Cognitive abilities or skills are supported by specific neuronal networks. For example, memory skills rely mainly on parts of the temporal lobes and parts of the frontal lobes (behind the forehead).

Non-limiting examples of cognitive abilities which may be boosted upon concomitant consumption of alcohol and at least one neurotransmitter or a precursor thereof include perception, attention, memory, motor skills, language, visual and spatial processing, logic and reasoning, auditory processing, processing speed and/or executive functions.

“Perception”, as used herein, is recognition and interpretation of sensory stimuli such as smell, touch, hearing, and the like.

“Attention”, as used herein, is the ability to sustain concentration on a particular object, action, or thought, the ability to ignore distractions and ability to manage competing demands. Attention also includes divided attention and sustained attention. Sustained attention is the ability to stay focused and on task for an extended period of time. Signs that sustained attention skills may be weak include jumping from project to project, and/or always being surrounded by unfinished projects. Divided attention is the ability to remember information while doing two things at once. Signs that divided attention skills may be weak include not being able to multitask or making frequent mistakes.

“Memory”, as used herein, is short-term/working memory and long-term memory. Working memory is the ability to hang on to information while being in the process of using it. Signs that working memory skills may be weak include having to read the directions again in the middle of a project, experiencing difficulty following multi-step directions, forgetting what was just said in a conversation. Long-term memory is the ability to hang on to, and access, stored information that was learned in the past. Signs that long-term memory skills may be weak include, for example, forgetting names, doing poorly on tests, forgetting known things.

“Motor skills”, as used herein, is the ability to mobilize muscles and body, and ability to manipulate objects.

“Language”, as used herein, is skills allowing a subject to translate sounds into words and generate verbal output.

“Visual and spatial processing”, as used herein, is the ability to process incoming visual stimuli, to understand spatial relationship between objects, and to visualize images and scenarios. Signs that visual processing skills may be weak include, for example, struggling to understand and/or remember what has been just read, following directions, and reading maps.

“Auditory processing”, as used herein, is the ability to analyze, blend, and segment sounds, and is a critical skill for successful reading. Signs that auditory processing skills may be weak include, for example, having difficulties in learning to read, or struggling with reading fluency or comprehension.

“Logic and reasoning”, as used herein, is the ability to reason, form ideas, and solve problems. Signs that logic and reasoning skills may be weak include frequently asking “What do I do next?”, or say “I don't get this,” struggling with math, feeling stuck or overwhelmed.

“Processing speed”, as used herein, is the ability to perform tasks quickly and accurately. Signs that processing speed is weak include the ongoing feeling that tasks are more difficult for oneself than for other people, taking a long time to complete tasks for school or work, frequently being the last one in a group to finish something.

“Executive functions”, as used herein, is abilities that enable goal-oriented behavior, such as the ability to plan, and execute a goal. Abilities that enable goal-oriented behavior include: flexibility in switching to the appropriate mental mode; theory of mind; anticipation and prediction based on pattern recognition; problem-solving; decision making; working memory or the capacity to hold and manipulate information in real time; emotional self-regulation or the ability to identify and manage one's own emotions for good performance; sequencing and prioritizing; and inhibition or the ability to withstand distraction, and internal urges.

The term “mental”, as defined herein, relates to the mind and its activity, i.e., occurring or experienced in the mind, or involving the process of thinking. Specifically, mental relates to the total emotional and intellectual responses of an individual to external reality. Mental also refers to intellectual activity as contrasted with emotional activity or with overt physical activity.

The terms “mental process” or “mental function”, as used herein, refers to all the things that individuals can do with their minds. These include perception, judgement memory, thinking (such as ideation, imagination, belief, reasoning and the like), volition, and emotion.

The term “emotion”, as used herein, is any conscious experience characterized by intense mental activity and a certain degree of pleasure or displeasure. Emotion, herein, is the affective aspect of consciousness, and is interchangeable with the terms “state of mind” and “feeling”. For example, anger or fear, which are subjectively experienced as strong feeling usually directed toward a specific object and typically accompanied by physiological and behavioral changes in the body. Emotions may sometimes be intertwined with mood, temperament, disposition, and motivation. In some embodiments, cognition is an aspect of emotion. Further, and non-limiting, examples of emotions or states of mind include, affection, anger, angst, anguish, annoyance, anticipation, anxiety, apathy, arousal, awe, boredom, confidence, contempt, courage, curiosity, depression, desire, despair, disappointment, disgust, distrust, ecstasy, embarrassment, empathy, enthusiasm, envy, euphoria, fear, frustration, gratitude, grief, guilt, happiness, hatred, hope, horror, hostility, humiliation, interest, jealousy, joy, loneliness, love, lust, outrage, panic, passion, pity, pleasure, pride, rage, regret, rejection, remorse, resentment, sadness, self-confidence, shame, shock, shyness, sorrow, suffering, surprise, trust, wonder, and worry.

In some embodiments, a contemplated method, by providing to a subject in need thereof an effective amount of at least one neurotransmitter or a precursor thereof upon alcohol consumption, imparts, enhances, elevates boosts and/or improves in the subject one or more of: concentration ability, focus, alertness, motivation, learning ability, pain processing, mood attention span, creativity, curiosity, physical and cognitive performance particularly under stress, ability to cope with negative mood, pursuing rewarding feelings of satisfaction and/or pleasure, euphoria, joy and happy feelings, relaxation, and optimism.

In an aspect, the present disclosure relates to the use of alcohol and at least one neurotransmitter or a precursor thereof as psychoactive means for imparting or bestowing to a subject a desired emotional and/or mental effect.

In some embodiments, a desired emotional and/or mental effect is selected from: euphoric feeling, elevated positive and social mood, joyfulness, satisfaction, pleasure, stress relief, relaxation, optimism, creativity, stimulation, blissfulness, sense of being rewarded.

In a further aspect, the present disclosure relates to the use of alcohol and at least one neurotransmitter or a precursor thereof as psychotropic means for improving a desired cognitive ability or skill in a subject.

In some embodiments, the desired cognitive ability is selected from: (i) higher motivation, e.g., to work, learn and take part in rewarding activities; (ii) improved concentration and focus; (iii) higher self-confidence; (iv) arousal; (v) wakefulness; (vi) elevated alertness; (vii) improved creativity and creative thinking (viii) curiosity and openness to new experiences; (ix) sense of self-fulfillment, self-contempt; (x) relaxation; (xi) improved capacity to switch attention efficiently between tasks; (xii) improved sociability and extroversion behavior; (xiii) sense of well-being; (xv) stress relief; and (xvi) ability to cope with negative mood.

In a further aspect, provided herein is a method for enhancing or strengthening a desired psychoactive effect and/or psychotropic affect brought upon, initiated, kicked off or triggered by alcohol consumption, the method comprising consuming alcohol together with one or more neurotransmitters and/or one or more neurotransmitter precursors, thereby enhancing or strengthening a desired psychoactive and/or psychotropic effect of alcohol consumption. Any of the desired psychoactive effects and/or psychotropic affects described herein may be enhanced or strengthened by a contemplated method.

In yet a further aspect, the present disclosure relates to a method for ameliorating, mitigating, reducing, lowering or nulling an adverse or negative effect associated with alcohol consumption, the method comprising consuming alcohol together with one or more neurotransmitters and/or neurotransmitter precursors, thereby ameliorating or reducing an adverse or negative effect of alcohol consumption. Negative effects of alcohol consumption which can be mitigated, lowered, reduced and even nulled are, for example, intoxication, sedative effects, dysphoria, anxiety, depression, despair, pessimism, negative mood, reduced psychomotor functioning and impaired or adversely affected cognitive abilities such as inhibition, attention control, and planning.

The term “administering”, as used in the context of some embodiments described herein, means providing, supplying, dispensing or giving. “Administer to a subject” in a context herein means providing a subject with, supplying, giving or dispensing to the subject.

Administration in a broad meaning, as sometimes applies in embodiments described herein, is further referred to as introduction of an active compound or of a formulation comprising it to a subject by a chosen route, for example introduction of a neurotransmitter and/or a neurotransmitter precursor, or a pharmaceutical composition comprising it. Administration of the active compound or pharmaceutical composition can be by any route selected from local or systemic administration as well known to one of skill in the art, and as appropriate for the particular condition.

In some embodiments, provision of an effective amount of at least one neurotransmitter or a precursor thereof and alcohol is by way of consuming, for example, eating, drinking, smoking or inhaling.

In some embodiments, oral administration is contemplated. In the context of these embodiment, a subject “treated” by a disclosed method is orally provided with alcohol and at least one neurotransmitter and/or at least one neurotransmitter precursor.

An effective amount is a quantity of a neurotransmitter and/or a precursor thereof or a formulation or food product comprising same, sufficient to achieve a desired effect in a subject being provided or treated with a method of the present disclosure.

In some embodiment, a disclosed method is applied by providing to a subject an alcoholic food product, supplemented or enriched with at least one neurotransmitter and/or at least one neurotransmitter precursor, for example, an enriched alcoholic beverage, as described herein in the preceding sections.

It shown herein, that in a Biphasic alcohol effects scale (BAES) test (described in Example 12 herein), participants who consumed enriched alcoholic beer containing tyrosine and caffeine, experienced significantly strengthened and prolonged stimulant and mood elevating effects as compared to consumption of a corresponding non-enriched beer. Moreover, consumption of the enriched beer significantly reduced the sedative effects, and for much longer time, as compared to the non-enriched beer.

Enhancement of the positive effects of alcohol may help reduce the risk of alcohol abuse. Motives of alcohol consumption are classically “enhancing motives” (drinking to enhance positive and social mood) and “coping motives” (drinking to cope with negative mood). While enhancing drinkers enjoy the positive effects of alcohol consumption, coping drinkers rely on its sedative effects, and are strongly exposed to risks of alcohol abuse and alcoholism. By enhancing alcohol's positive effects and reducing or suppressing its sedative effects, a contemplated method described herein may be useful in reducing risks of alcohol abuse amongst coping drinkers.

In some embodiments, a contemplated method is designed to promote at least the levels of dopamine, endorphin, norepinephrine and/or epinephrine in the brain. To this end, alcoholic products enriched with one or more of these neurotransmitters and/or their precursors are employed. For example, alcoholic products containing L-phenylalanine (Phe), L-tyrosine (Tyr) and/or levodopa (L-dopa), which are the precursors of dopamine and other catecholamines such as norepinephrine and epinephrine, may be used, in accordance with a disclosed method, for elevating production of DA, endorphin, norepinephrine and/or epinephrine in a subject upon alcohol consumption.

A disclosed method may be referred to herein as a “psychoactive method” practiced in order to boost, initiate, exert, promote, improve or enhance positive psychoactive effects in a subject. A disclosed method may further be referred to herein as a “psychotropic method” practiced in order to boost, initiate, exert, promote, improve or enhance positive psychotropic affects in a subject.

In some embodiment, a disclosed method comprises administration, as defined herein, of alcohol dopamine and/or one or more dopamine precursors.

In some embodiments, a disclosed method comprises administration of alcohol and at least one dopamine precursor.

In some embodiments, a contemplated method comprises administration of an enriched alcoholic beverage, as defined herein, comprising at least one DA precursor selected from Phe, Tyr or L-dopa. In exemplary embodiments, the DA precursor is Tyr in any of the amounts disclosed herein.

In some embodiments, a contemplated method may comprise the provision to a subject of alcohol, at least one neurotransmitter and/or a precursor thereof, and at least one psychostimulant substance as defined herein. In some embodiments, the psychostimulant substance is caffeine provided in any of the amounts disclosed herein.

It is to be understood that the invention as described and discussed in the foregoing and in following sections of the description is not necessarily limited in its application to the details set forth in the present description.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “10 μm” is intended to mean “about 10 μm”.

The term “about” as used herein means within an acceptable error range for a particular value as determined by one of ordinary skill in the art, which will depend in part on how the value is measured or determined, i.e., the limitations of the measurement system. For example, “about” can mean a range of up to 10%, more preferably up to 5%, and still more preferably up to 1% of a given value.

The terms “comprises”, “comprising”, “includes”, “including”, “having” and their conjugates mean “including but not limited to”.

The term “consisting of” means “including and limited to”.

The term “consisting essentially of” means that the compositions, for example, enriched alcoholic beverages, may include additional ingredients but only if the additional ingredients do not materially alter the basic and novel characteristics of the claimed composition.

As used herein, the singular form “a”, “an” and “the” include plural references unless the context clearly dictates otherwise. For example, the term “a compound” or “at least one compound” may include a plurality of compounds, including mixtures thereof. It is appreciated that certain features of the present disclosure, which are, for clarity, described in the context of separate embodiments, may also be provided in combination in a single embodiment. Conversely, various features of the disclosure, which are, for brevity, described in the context of a single embodiment, may also be provided separately or in any suitable subcombination or as suitable in any other embodiment described herein. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the present disclosure in a non-limiting fashion. Generally, the techniques used herein are thoroughly explained in the literature and are believed to be well known in the art.

### Example 1

### Preparation of Beer Enriched with Tyrosine and Caffeine

Beer enriched with tyrosine and caffeine was prepared using a protocol comprising the following steps:

(i) grinding barley;

(ii) immersing the grinded barley in water at a temperature of 50-75° C. for about an hour;

(iii) adding caffeine;

(iv) transferring the mixture of grinded barley and caffeine to a boiler;

(v) adding hops to the mixture in the boiler, and boiling the mixture for about an hour;

(vi) transferring the boiling mass (herein referred to as the “must”) into a fermentation tank via a heat exchanger apparatus that cools the must form 100° C. to about 20° C.;

(v) adding yeasts to the cooled must;

(vi) fermenting the must for a period of 14 to 16 days so as to obtain the beer;

(vii) transferring the fermented mass into a second tank (herein also termed “intermediate tank”);

(viii) adding tyrosine and, optionally, glucose and to the fermented beer; and

(ix) transferring the beer to bottles, cans or barrels and storing for a second fermentation for a period of 12-40 days at 18° C.

The enriched beer was unfiltered and contained 5.5% alcohol by volume, 40 mg tyrosine and 35 mg caffeine, per 100 ml.

### Example 2

### Beer Enriched with Tyrosine and Caffeine

A further recipe for brewing beer enriched with tyrosine and caffeine comprises the following steps:

(i) milling malted barley;

(ii) mashing: transferring milled malted barley into mash tun, adding water and heating to a temperature of from about 50° C. to about 75° C. for about 1-2 hour;

(iii) lautering: separating the wort (the malty liquid that was extracted during mashing) from the grains;

(iv) adding tyrosine (from about 100 mg/L to about 5000 mg/L);

(v) boiling the wort;

(vi) adding hops and boiling the mixture for about 45-90 minutes;

(vii) separating hopped wort from solid particles;

(viii) cooling the wort to fermentation temperature (about 4-25° C.) and aerating it;

(ix) adding brewer's yeasts and fermenting the wort for several weeks;

(x) conditioning/maturing: separating fermented beer from dead yeasts and other debris and transferring it into a conditioning tank for a period of several weeks to several months;

(xi) filtering the beer;

(xii) adding caffeine (from about 10 mg/L to about 700 mg/L);

(xiii) heating the beer to 70° C. for pasteurization (elimination of harmful bacteria) and to fully dissolve added tyrosine and caffeine; and

(xiv) packaging the enriched beer (i.e., bottling, canning and/or transferring to a barrel), and cooling for storage.

### Example 3

### Alcopop (“Hard Lemonade”) Enriched with Tyrosine and Caffeine

Hard lemonade, an alcopop, enriched with tyrosine and caffeine is prepared according to the following steps:

(i) grinding mint leaves with brown sugar;

(ii) adding boiling water;

(iii) adding tyrosine (from about 100 mg/L to about 5000 mg/L), and stirring the tyrosine-containing mixture;

(iv) adding the following ingredients to the above mixture: water (at room temperature), a spirit with at least 40% alcohol (e.g., vodka), and freshly squeezed lemon juice with pulp, in a ratio mixture:water:vodka:lemon juice of about 1:5:4:2;

(v) adding caffeine (from about 10 mg/L to about 700 mg/L); and

(vi) bottling and/or canning the enriched alcopop, and cooling for storage.

### Example 4

### White Wine Enriched with Tyrosine and Caffeine

White wine enriched with tyrosine and caffeine is prepared according to the following steps:

(i) crushing, pressing and extracting the grape juice (wort) of white-flesh grapes (so as to separate it from skins, seeds and other solids);

(ii) clearing the wort to remove lees, for example, by settling or by other methods;

(iii) adding tyrosine (from about 100 mg/L to about 5000 mg/L);

(iv) optionally, adding wine yeasts to the wort in addition to the existing natural yeasts;

(v) fermenting the wort in fermentation tanks for several weeks at a temperature of 12-37° C.;

(vi) eliminating fermentation, if desired.

(vii) clearing and refining: separating the fermented wine from dead yeasts and other debris, for example, by filtration or by other means;

(viii) adding caffeine (from about 10 mg/L to about 700 mg/L; and

(ix) bottling the enriched wine.

### Example 5

### Red Wine Enriched with Tyrosine and Caffeine

Red wine enriched with tyrosine and caffeine is prepared according to the following steps:

(i) destemming and crushing grapes to create a must (i.e., a mixture of grapes, skins, juice and seeds);

(ii) settling the must for pre-fermentation maceration at a temperature of about 10° C. for a period of 1 to 4 days;

(iii) adding tyrosine (from about 100 mg/L to about 5000 mg/L);

(iv) optionally, adding wine yeasts to the wort in addition to the existing natural yeasts;

(v) fermenting the must in fermentation tanks for several weeks at a temperature of 12-37° C. During fermentation, the contact between skins and liquid phase can be maximized by a desired mixing method;

(vi) extracting the juice from the must by pressing and separating it from remaining solids;

(vii) allowing for malolactic fermentation;

(viii) racking: decanting off lees (dead yeasts and other solids) and providing sulfur dioxide preservative to avoid oxidation and bacterial spoilage;

(ix) aging/maturing the wine for a desired period;

(x) clearing and refining, for example, by filtration or by other means;

(xi) adding caffeine (from about 10 mg/L to about 750 mg/L; and

(xii) bottling the enriched wine.

### Example 6

### Cider Enriched with Tyrosine and Caffeine

“Hard cider” enriched with tyrosine and caffeine is prepared according to the following steps:

(i) crushing and pressing apples to acquire must (apple juice) from pomace (mixture of solid remains of pressed apples, skins, juice and seeds);

(i) adding tyrosine to concentrations of 100 mg/L-5 gr/L;

(iii) optionally, adding cider yeasts to the must, in addition to existing natural yeasts;

(iv) fermenting the must in fermentation tanks for several weeks at a temperature of 4-16° C.;

(v) racking: siphoning the liquor into new vats, separating it from dead yeasts and undesirable materials;

(vi) aging/maturing the cider for a desired period;

(vii) adding caffeine to concentrations of 10 mg/L to about 750 mg/L; and

(viii) packaging the enriched cider (i.e., bottling, canning and/or transferring to a barrel), and cooling for storage.

### Example 7

### Single Malt Whisky Enriched with Tyrosine and Caffeine

Single Malt whisky enriched with tyrosine and caffeine is prepared according to the following steps:

(i) milling malted barley;

(ii) mashing: transferring milled malted barley into mash tun, adding hot water in three stages and elevating the temperature (beginning with approx. 60° C., then approx. 75° C. and, finally, approx. 90° C.). Wort (the malty liquid that is extracted during mashing) is transferred into a washback;

(iii) cooling the wort to fermentation temperature (about 4-25° C.);

(iv) adding whisky yeasts;

(v) fermenting the cooled wort for up to several days to receive the “wash” (i.e., insipid beverage);

(vi) distilling the wash twice using stills: first using wash stills to obtain “low wine”, and a second time using spirit stills to obtain the final “new-make” spirit (unaged whisky). During the second distillation, the more volatile compounds which distil off first (foreshots), and the final stage where more oily compounds are vaporized, are both channeled off so that only the pure center cut is collected;

(vii) optionally, diluting the new-make prior to aging;

(viii) transferring new-make to wooden casks and aging for several years;

(ix) chill filtrating: precipitating and filtering to remove fatty acid esters;

(x) adding tyrosine (final concentration 100 mg/L-5 gr/L);

(xi) adding caffeine (final concentrations 10 mg/L to about 750 mg/L); and

(xii) bottling the enriched whisky.

### Example 8

### Vodka Enriched with Tyrosine and Caffeine

Vodka enriched with tyrosine and caffeine is prepared according to the following steps:

(i) mashing desired starchy ingredient (potatoes, milled grains, etc.), adding water and boiling;

(ii) cooling the mash to enzyme activity temperatures;

(iii) adding starch-breaking enzymes, such as amylase;

(iv) allowing enzyme activity to break down starch within mash to small sugars;

(v) adding vodka yeasts;

(vi) fermenting the starch-broken mash for several days in yeast-activity temperatures (about 4-25° C.) to obtain the wash;

(vii) distilling the wash using standard methods to obtain raw spirit of ˜60% alcohol by volume (ABV). Separating and discarding products of the first and last distillation stages (i.e., first 5% and final 35% of the whole distillation process);

(viii) rectifying the raw spirit to neutral spirit of ˜96% ABV;

(ix) preparing demineralized water solution containing tyrosine (80-585 mg/L) and caffeine (15-670 mg/L), heating to ˜80° C. until homogenous, and then cooling to ambient temperature;

(x) adding the demineralized water solution to the neutral spirit to reduce ABV to ˜40%;

(xi) filtering the obtained enriched vodka using standard methods; and

(xii) bottling the enriched vodka.

### Example 9

### Coffee Liqueur Enriched with Tyrosine and Caffeine

Coffee liqueur enriched with tyrosine and caffeine is prepared according to the following steps:

(i) combining 3 cups of sugar, 2 cups of water and a tablespoon of vanilla extract in a saucepan;

(ii) boiling and stirring for approx. 30 minutes until volume is reduced by half;

(iii) adding 100-400 mg tyrosine and about 100 gr instant coffee, stirring until homogenous and allowing the syrup mixture to cool;

(iv) combining the syrup with 750 ml 40% ABV vodka in a closed 1-liter bottle, and shaking well;

(v) optionally, adding up to 180 mg caffeine;

(vi) settling for 10 days in a cool, dark place;

(vii) Straining/filtering the enriched liqueur; and

(viii) bottling the enriched liqueur.

### Example 10

### General Procedure for Production of Wine from Fruit Juice

Extracted juices from harvested fruits were used as fermentable base material for wine making. To prevent the growth of undesirable microorganisms, the juice extracts were pasteurized. All utensils, tools, and equipment that came into contact with the wine in making, were sterilized and rinsed thoroughly. No chemicals were used in the preparation of the must. Sufficient amounts of yeast nutrients such as sugar were added for yeast growth. The pH of the must was adjusted, and sufficient sugar was added where needed to produce 9%-11% percent alcohol in the finished wine. Optionally, a small amount of quinine solution was added to provide bite and flavor to the finished wine. The yeasts used were either purchased and activated according to the manufacturer's directions, or reserved from wines made, which were kept under refrigeration and used for subsequent wine production. All the wine-making stages: first and second fermentations, raking, storage and aging, were carried out in an air-conditioned room so that constant temperatures could be maintained. Finished wines were bottled and/or canned, pasteurized, and cooled for storage and/or to age in the bottles.

### Example 11

### Preparation of Flavored Non-Carbonated Apple Wine Beverages

Harvested apples were crushed and pressed to acquire apple juice, and apple wine was prepared as described in Example 10 above. To the apple wine produced, tyrosine, caffeine, and flavorants were added to obtain 3 flavored non-carbonated apple wines-based beverages: grapefruit, bitter lemon and mint. The flavorants used were natural extracts or juices of citrus and mint. The apple wine base material was 87%-95% by volume of total beverage. The beverages were pasteurized and transferred to 150 ml aluminum cans and stored until used. The ingredients and amount thereof used in the preparation of the apple wine-based beverages are further disclosed in the Table 1 below.

TABLE 1
Exemplary apple wine-based beverages

| | Mint flavored apple | Grapefruit flavored | Bitter lemon flavored |
|---|---|---|---|
| Ingredients | wine drink | apple wine drink | apple wine drink |
| Tyrosine (gr/L) | 0.35-0.45 | 0.35-0.45 | 0.35-0.45 |
| Caffeine (mg/L) | 140-150 | 140-150 | 140-150 |
| Sugar (gr/L) | 40-55 | 40-55 | 40-55 |
| Citric acid (gr/L) | 1.0-1.5 | 3.0-3.3 | 3.0-3.3 |
| Flavorant (gr/L) | 4.0-4.3 | 3.5-3.7 | 6.9-7.1 |
| Ascorbic acid (gr/L) | 0.15-0.25 | | 0.15-0.25 |
| Alcohol (% v/v) | 10 | 10 | 10 |
| Clarity | cloudy drink, | cloudy drink, | cloudy drink, |
| | without sediments | without sediments | without sediments |
| Color | white/light yellow | pink | colorless/light straw |

All three beverages tasted fresh, were refreshing, characteristic for used ingredients, without foreign aftertaste, and presented the typical flavors for the ingredients used.

### Example 12

### Biphasic Alcohol Effects Scale (BAES) Study

The stimulant and sedative effects caused by consuming a beer enriched with a neurotransmitter precursor such as tyrosine and, optionally, a psychostimulant substance such as caffeine, versus the stimulant and sedative effects exerted by a corresponding non-enriched beer was assessed in self-reporting participants using a biphasic alcohol effects scale (BAES)-based questionnaire. The BAES is a reliable and valid systematic 14-item self-report scale designed to measure stimulant and sedative effects of alcohol as separate and distinct constructs. BAES and a brief version thereof (B-BAES) comprising only 6 items are described, for example, in Rueger and King, 2013 (Alcohol Clin Exp Res. 37(3): 470-476; and references cited therein).

Seven items comprise the stimulant subscale of the BAES (elated, energized, excited, stimulated, talkative, up, vigorous), and another seven items comprise the sedative subscale (difficulty in concentrating, down, heavy head, inactive, sedated, slow thoughts, sluggish). The BAES test demonstrates strong psychometric properties, including high internal consistency, reliability and a four-factor structure reflecting the distinctness of the stimulant and sedative constructs during both the ascending and descending limbs of the breath alcohol concentration (BrAC) curve. This four-factor structure is invariant to dose, drinking history, and sex, and demonstrates robustness to an instructional set that does not disclose the content of the alcoholic beverage (see, for example, Rueger et al., 2009, Alcohol Clin Exp Res. 33:916-924).

The BAES study of alcohol effects in accordance with the disclosed study had multiple assessments in one session. For example, the BAES questionnaire was administered before alcohol was consumed and then the assessment was repeated 3-4 more times after consumption to capture rising and declining BrAC limb effects.

In the BAES questionnaire exemplified herein, each participant was presented with 14 items in alphabetical order, 7 of which describe sensations (feelings, perception) associated with stimulant effects (elevated mood (high spirit), energy, excitement, stimulated, talkativeness, vigorous and vitality), and 7 items describing sensations (feelings, perception) associated with sedative effects (difficulty in concentrating, down feeling, heavy head, heavy body, sedative, slow thinking, sluggishness). Each participant had to assign, next to each of these 14 items, a number in the range from 0 to 10 indicating how closely or how well a certain item expresses, corresponds or describes his/her current feeling/sensation, where “0” indicates “not at all describing” or “irrelevant”, and “10” indicates “exactly (or very much) describing”.

The numerical indications of the stimulant and sedative effects were averaged to obtain two numerical values designated STIM and SED, respectively.

### Participants

Healthy nonalcoholic 198 social drinkers participated in the study. Participants were recruited from the wider community via advertising placed, for example, in web sites. Each participant's eligibility was subject to the following criteria: (i) aged 18-60 years; (ii) no history of alcohol addiction; (iii) not taking medication having a stimulative or sedative action; (iv) moderate to low regular consumption of alcohol.

The gender split was equal across both groups. All participants were requested to refrain from alcohol consumption at least 24 hours before the study.

### Study Design

The study was a 2-session (2 days) study, wherein the second session (day) was at least a week apart from the first session. Participants were randomly supplied with either one of: (i) beer having 5.2% alcohol and enriched with tyrosine and caffeine; or (ii) a corresponding non-enriched beer. The study was double-blinded, and the participants did not know whether they were supplied with alcoholic beverage (i) or (ii).

In each session, participants first filled a BAES questionnaire to set up a baseline assessment, followed (about 5 minutes later) by the consumption of either enriched beer (i) or non-enriched beer (ii). For each participant, the amount of beer provided was calculated so as to reach blood alcohol level of 0.35 gr per kg body weight. For example, for a participant weighting 75 kg, 26.25 gr of alcohol are required in order to reach the predetermined blood alcohol concentration, thus this participant was supplied with 504.8 ml beer having 5.2% alcohol.

The total amount of alcohol for a participant was divided into 5 equal portions, and the participant was requested to consume each portion within 2 minutes (overall, 10 min for consuming the whole amount of alcoholic beverage). Immediately after the last portion of beverage was fully consumed, time point zero (T=0) was set. Participant were requested to fill a BAES questionnaire at T=30 min, T=60 min and at T=120 min.

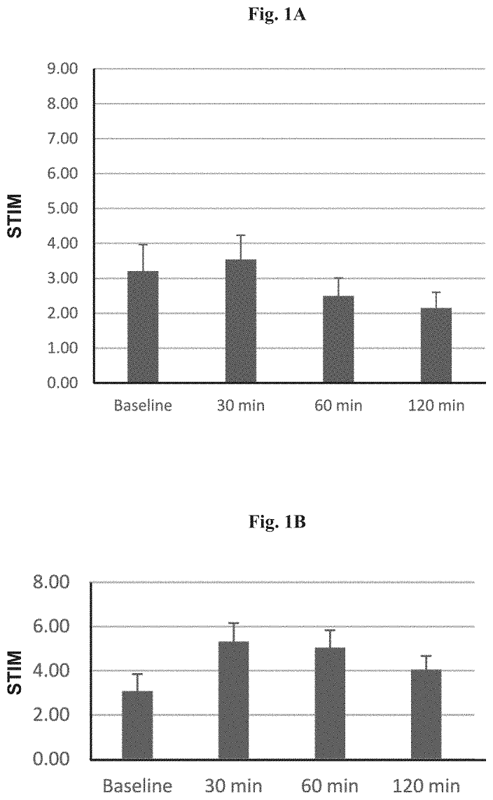

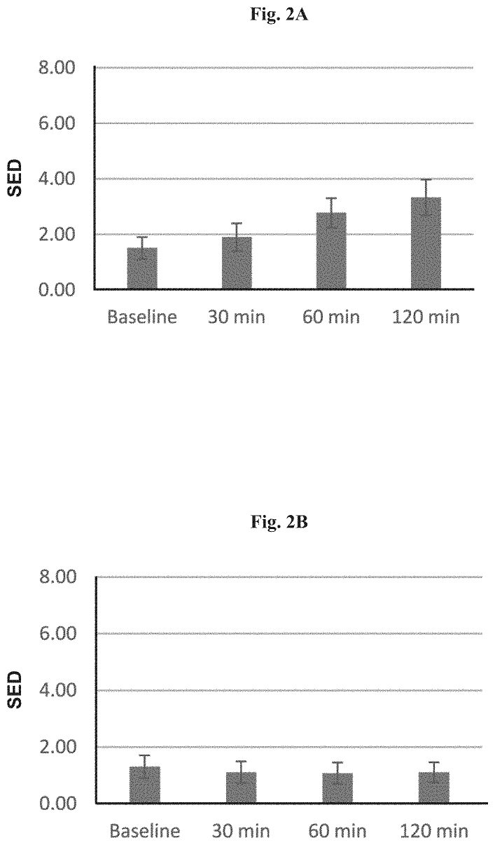

Answers obtained from all BAES questionnaires were gathered, averaged (standard deviation was calculated) to obtain the STIM and SED values, and presented graphically. The results are shown in FIGS. 1A-1B and 2A-2B and presented in Tables 2 and 3.

TABLE 2
BAES study for non-enriched (regular) beer
Regular (non-enriched) Beer

| | STIM | SD | SED | SD |
|---|---|---|---|---|
| Baseline | 3.20 | 0.76 | 1.51 | 0.39 |
| 30 min | 3.54 | 0.69 | 1.90 | 0.50 |
| 60 min | 2.49 | 0.52 | 2.78 | 0.53 |
| 120 min | 2.15 | 0.45 | 3.33 | 0.64 |

TABLE 3
BAES study for beer enriched with tyrosine and caffeine
Enriched Beer

| | STIM | SD | SED | SD |
|---|---|---|---|---|
| Baseline | 3.08 | 0.77 | 1.30 | 0.40 |
| 30 min | 5.32 | 0.83 | 1.10 | 0.38 |
| 60 min | 5.04 | 0.79 | 1.07 | 0.38 |
| 120 min | 4.05 | 0.61 | 1.10 | 0.35 |

As seen in Table 2, and FIGS. 1A and 2A, 30 minutes after the non-enriched beer was consumed, participants experienced slight stimulant and sedative effects. Sixty minutes (T=60) and 120 minutes (T=120) after consumption, the sedative effects increased while stimulant effects decreased even below baseline levels.

However, as seen in Table 3, and FIGS. 1B and 2B, 30 minutes after consuming the enriched beer, significant elevation of stimulant effects was demonstrated, along with a slight rise in sedative effects. Sixty minutes (T=60) and 120 minutes (T=120) after consumption of the enriched beer, the stimulant effects gradually regressed or decreased, but they were still higher compared to consumption of non-enriched beer. No significant change was seen in the sedative effects, namely, they remained low even after 120 min.

It is shown herein that consumption of regular alcoholic beer causes an initial rise to stimulant and uplifting effects followed by a sharp decline to below starting value. This decline is correlated with gradual rise to sedative and depressing effects over time. In comparison, consumption of beer enriched with Tyr and caffeine generated a significant increase to uplifting effects which was kept well above starting value for over two hours after consumption, while initial sedative values remained constant and showed no incline over time.

### Example 13

### Testing the Effects on Cognitive Abilities of Consuming Alcohol Combined with a Neurotransmitter Precursor

The aim of this study is to assess the effects of combined consumption of alcohol, a neurotransmitter precursor such as tyrosine and, optionally, a psychostimulant substance such as caffeine across a wide range of key cognitive processes and abilities. In addition, this study compares the relative sensitivity of different cognitive abilities to the presence and absence of a neurotransmitter precursor and, optionally, a psychostimulant substance in an alcoholic food product.

The tests employed in this study are based on known tests designed to assess the immediate adverse effects of alcohol on various aspects of cognitive functioning, as described, for example, in Matthew et al., (2012) (PLOS ONE, 7: e50977; and references cited therein). A test battery is exemplified herein, comprised of three psychometric tests repeated during a session. The tests employed are indices of a range of key cognitive processes that have previously been shown to be sensitive to the effects of alcohol intoxication, for example, speed of information processing, divided attention, problem solving, working memory, response inhibition and cognitive flexibility, and psychomotor functioning.

### Participants

Written consent is obtained from each of the participants. Participants are drawn from the wider community via advertising placed, for example, in local newspapers and community web sites. Each participant's eligibility is subjected to the following criteria: (i) age 18-45 years; (ii) no major medical or psychiatric conditions; (iii) no visual disorders; (iv) no dependence on any substance (excluding nicotine); (v) not taking medication having a stimulative or sedative action; (vi) moderate regular consumption of alcohol; and (vii) had consumed at least five alcoholic beverages on at least one occasion in the past month. The age range is chosen to ensure that the participants are old enough to be of legal drinking age, but young enough to ensure that they are unlikely to be affected by any deleterious effects of ageing upon cognitive abilities. Criterion (vii) is chosen to ensure that the participants had prior experience with ingesting and functioning under the dose sizes of alcohol employed in the experiment. It is important that the participants are all ‘experienced’ drinkers. The gender split is equal across both groups. All participants are requested to refrain from alcohol consumption at least 24 hours before the study.

Participants are randomly supplied with either one of: (i) an alcoholic beverage, e.g., beer, enriched with a neurotransmitter precursor (e.g., tyrosine) and, optionally, with a psychostimulant substance (e.g., caffeine); or (ii) a corresponding non-enriched alcoholic beverage.

### Cognitive Abilities Tests

Tree cognitive ability tests are employed: (i) inspection time (IT); (ii) self-ordered pointing task (SOPT); and (iii) sustained attention to response task (SART). For all tests, computerized versions of the tasks may be employed as described, for example, in Burns and Nettelbeck (2003) (Intelligence, 31: 237-255) and Robertson et al. (1997) (Neuropsychologia, 35: 747-758), or generated using MatLab.

(i) Inspection Time (IT) Test

Inspection time (IT) is a measure of speed of information processing. Inspection time is motor free, i.e., it does not require a speeded response on behalf of a participant, rather it measures the minimum display-time necessary for a participant to make two-alternative forced-choice decision (see Deary et al., 2004, Neuroimage 22: 1466-1479).

The test is as follows: a shape or a design version, e.g., an inverted U shape having one “leg” shorter than the other, is flickering or repeatedly presented on the computer screen at varying rate. The participant needs to elect/indicate which is the side of the shorter leg. The shortest display-time needed for a participant to correctly identify the shorter leg with clear statistical significance (about 90% of times) is scored. The participant is advised prior to testing that the purpose of the test is to identify the correct forced-choice decision (short leg) but there is no time constraint.

(ii) The Self-Ordered Pointing Task (SOPT) Test

The self-ordered pointing task (SOPT) is a measure of working memory function. Performance on the task requires participants to hold visual information in short-term storage while executing a response strategy and continuously monitoring performance.

The test is as follows: participants are presented with, for example, 10, 12 and 16 design versions (images) in a continuously changing order relative to each other with each design version repeated three times per test run. Prior to each change of order, the participant is asked to select a different design version and avoid from choosing the same version again in subsequent presentations. The participant is practicing this test several times, each time having to handle increasing number of images changing their location on the screen relative to the other images. The number of incorrect image selections by the participant is scored. Prior to the test, a participant is advised that image selection is to be performed at intermediate rate, namely not too slow or too fast.

(iii) The Sustained Attention to Response Task (SART) Test

The sustained attention to response task (SART) is a measure of response inhibition and cognitive flexibility. Participants are required to respond quickly to a commonly occurring set of stimuli but withhold responding to a rarely occurring target stimulus. Performance on the task involves regions of the prefrontal cortex associated with inhibitory control, performance monitoring and error processing.

The test is as follows: shapes, e.g., numbers, are flickering or repeatedly presented on the computer screen at constant rate. The participant is asked to press a certain key on the keyboard after each presentation, unless a predetermined shape appears on the screen, in which case the participant should refrain for pressing the keyboard. The number of faults (namely, pressing the key when shouldn't and not pressing it when should) is scored. Prior to the test, a participant is advised that accuracy is important, while key pressing is to be done at minimum time.

Each of tests (i)-(iii) lasts about 5 minutes. All three tests are conducted sequentially, in a predetermined and fixed order, over a total time of 15-20 minutes. Tests are conducted double blindingly.

### Study Schedule

Each study is a 3 days study, wherein Day 1 is a familiarization session, and alcohol manipulation is applied on the second and third days as follows:

Day 1: acquaintanceship. Participants are first screened for drug and alcohol use, then presented with the tests and practice them, for simulation, three times in a row with 1-hour interval between each tests sequence.

Optionally, brief intellectual ability (BIA) scales are taken for each participant. This measure may comprise, for example, three tests from the Woodcock-Johnson III (Woodcock et al., (2001) Woodcock-Johnson III. Itasca, Ill.: Riverside): Verbal Comprehension (crystallized ability), Concept Formation (fluid ability), and Visual Matching (perceptual speed). It takes approximately 15-20 min to complete. The BIA is conducted once only on the first day in order to gauge each participant's level of intellectual ability.

Day 2: participants (after being screened for drug and alcohol use) first practice the sequence of tests and then conduct them sequentially for scores at least about 30 minutes before the actual study commences, in order to obtain reference data (i.e., baseline record or control data) for each participant, before alcohol consumption. About 10 minutes before the first test in the battery is taken, each participant in groups (i) and (ii) is provided either with five 100 ml portions of beer enriched, for example, with tyrosine and, optionally, caffeine, or the corresponding non-enriched beer, respectively, and instructed to consume each portion in 2 minutes such that after all 5 portions are consumed, blood alcohol level reaches 3.5 gr per kg body weight.

Immediately after the last portion of alcohol is consumed, time point zero (T=0) is set. At T=30 min and at T=60 min the battery of 3 tests is conducted by each participant.

Day 3: at least 48 hours after Day 1. Study is performed exactly as in Day 2, except the participants will consume the beer they did not consume on Day 2 (e.g., participants who were provided with enriched beer on Day 2, will be provided with non-enriched beer on Day 3).

Scores for all three tests will be statistically analyzed so as to obtain relevant results regarding the influence or effects on cognitive abilities exerted by the enriched beer as compared to the non-enriched beer.

Further cognitive ability tests and/or assessments that may be used to assess and/or quantify the effects of consuming alcohol together with a neurotransmitter and/or a precursor thereof on cognitive, mental and/or emotional abilities include, for example, the following tests:

The Traveling Salesperson Problem (TSP) Test.

The traveling salesperson problem (TSP) (Dry et al., 2006, J. Problem Solving 1: 20-32; 51) is a measure of strategic problem solving. Solving a TSP requires participants to continuously monitor their performance whilst making sequential decisions subject to multiple interacting constraints.

The Useful Field of View (UFOV) Test.

The useful field of view test (UFOV) (Ball et al., 1988, J. the Optical Society of America—A 5: 2210-2219; Goode et al., 1998, J. Clinical Psychology in Medical Settings 5: 425-440), is a measure of processing speed and divided visual attention. Task performance is reliant upon both the integrity of the viewer's visuo-sensory input, as well as higher-level cognitive functions.

Each of the tasks taken in these tests have been widely employed in the literature, is well-known and easily accessible. Participants require no special prior knowledge to perform the tasks and are able to perform them with a minimum of instructions. Each of the tasks can be completed in a short period of time (about 10 minutes) ensuring that a battery of tests can be completed within a time-period that minimizes any variability associated with rising and falling blood alcohol levels.

### Example 14

### Testing the Psychoactive Effects of Consuming Alcohol Combined with a Neurotransmitter Precursor

The aim of this study is to assess the psychoactive effects of combined consumption of alcohol, a neurotransmitter precursor such as tyrosine and, optionally, a psychostimulant substance such as caffeine. The tests employed in this study are based on known tests designed to assess the immediate adverse effects of alcohol on various aspects of cognitive functioning, accompanied by questionnaires presenting questions for evaluating the psychoactive effects and personal experience.

### Participants

Healthy nonalcoholic social drinkers between the ages of 21 and 31 years are drawn from the campus student of the Ben-Gurion university via advertisement displayed and distributed in the campus by the university's mail system and by social medias. Each participant's eligibility is subjected to the following criteria: (i) not currently pregnant or lactating; (ii) no major medical or psychiatric conditions; (iii) a negative urine drug screen (amphetamines, barbiturates, opiates, cocaine); (iv) no dependence, currently or in the past, on any substance (excluding nicotine); (v) not taking medication having a stimulative or sedative action; and (vi) moderate to low regular alcohol consumption.

Participants are assigned with randomized numeric codes that are used during collection and analysis of the data, while being evenly divided between genders with no bias or discrimination for any race or ethnicity. Written consent is obtained from each of the participants. All participants are compensated for their time.

At least the following tests and/or assessments are conducted:

Biphasic Alcohol Effects Scale (BAES).

A reliable and valid 14-item measure of alcohol's acute stimulant and sedative effects. An exemplary BAES test is described in Example 1 herein.

Profile of Mood States (POMS).

The POMS questionnaire is a standard validated psychological rating scale used to assess transient, distinct mood states. The version of the questionnaire used is the POMS 2 for adults aged 18 years and older, available as full-length (65 items) and short versions (35 items). The questionnaires contain a series of descriptive words/statements that describe feelings people have. Participants self-report on each of the items assessed using a 5-point Likert scale (a psychometric scale commonly used in surveys and questionnaires), ranging from 0 (Not at all), 1 (A little) 2 (Moderately), 3 (Quite a bit), to 4 (Extremely). Mood states are interpreted though 6 mood domains: tension or anxiety (9 items), depression or dejection (15 items), anger or hostility (12 items), vigor or activity (8 items), fatigue or inertia (7 items), and confusion or bewilderment (7 items). A total mood disturbance (TMD) score is calculated by summing the totals for the negative subscales (tension, depression, fatigue, confusion, anger) and then subtracting the totals for the positive subscales (vigor and esteem-related affect). Completion of the assessment takes 5-15 minutes, depending on the form.

Inspection Time (IT).

A computerized task that measures the speed of information processing that can be estimated in as little as 5 min. IT measures the minimum display-time necessary for a participant to make a two-alternative forced-choice decision (see Example 2 above).

Visual Analog Scale (VAS).

A psychometric scale that is generally used in pain scale surveys to understand varying degrees of pain experienced by a patient. In the current survey it is used for measuring characteristics and attitude related to alcohol consumption (e.g., enriched alcoholic beverage or corresponding non-enriched alcoholic beverage) across a range of continuous values. It's comparable to other linear scales like the Likert scale in terms of the results and the sensitivity of performance.

Buss-Perry Aggression Questionnaire (BPAQ).

A 29-item instrument that measures four factors: physical aggression, verbal aggression, anger, and hostility. The aggression questionnaire developed by Buss and Perry is a widely used measure of aggression in research and in applied settings. Buss-Perry Aggression Questionnaire is available, e.g., on https://www.researchgate.net/publication/301934203_Buss-Perry_Aggression_Questionnaire_Testing_Alternative_Measurement_Models_With_Assaultive_Misdemeanor_Offenders.

### Study Design

This study is a single-blind, randomized, cross-over study that includes two laboratory visits, at least one week apart. In order to set a unified physiological state, prior to each visit, participants are instructed to refrain from alcohol for at least 24 hours as well as from food, caffeine, and nicotine on the morning of testing. Participants arrive at 11:00 AM and are given a low-fat light lunch (juice, sandwich, pretzels). Then, participants undergo a set of baseline assessments, followed by the consumption of either a beer enriched with a neurotransmitter precursor such as tyrosine and, optionally, a psychostimulant substance such as caffeine, or a corresponding on-enriched beer (a 50 ml portion every 15 seconds), in a randomized manner, and are followed for 3 hours post consumption. Each visit is estimated to last about 5 hours.

First Visit.

Baseline assessments are performed for each participant based on one or more of the following tests or assessments:

(a) clinical interview using a structured diagnostic interview including demographic characteristics;

(b) a short version of the profile of mood states (POMS);

(c) Buss-Perry aggression questionnaire (BPAQ);

(d) inspection time (IT) computerized task;

(e) blood pressure measurement; and

(f) heart rate measurement.

Then, enriched and non-enriched alcoholic beverages are consumed as detailed above in Examples 1 and 2.

Second Visit.

Some of the tests of the first visit are repeated in order to re-evaluate the participants' baseline assessment. After baseline assessment, alcoholic beverages are consumed as detailed above.

Post consumption assessments for both visits is based on one or more of the following tests:

1. BPAQ, performed 15 minutes and 2 hours post consumption;

2. BAES, performed every 30 minutes;

3. POMS, performed 15 minutes, 30 minutes, 1 hour and 2 hours post consumption;

4. IT performed 15 minutes and 2 hours post consumption; and

5. VAS questions performed every 30 minutes.

In addition, blood pressure and heart rate are assessed every 30 minutes.

The primary outcome measures will be the subjective alcohol effects (BAES) and mood states (POMS) at 30 and 20 minutes post-consumption, as compared to baseline levels. Secondary outcome measures will be changes in IT performance, aggression levels, alcohol craving levels, and physiological parameters.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

What is claimed is:

1. An alcoholic food product comprising an edible base material, alcohol, and at least one of a neurotransmitter or a neurotransmitter precursor, wherein the edible base material is a liquid, solid or semi-solid edible substance.

2. The alcoholic food product of claim 1, wherein the edible base liquid substance is a base beverage selected from the group consisting of a natural or artificially flavored fruit juice, vegetable juice, fruit syrup, concentrate or nectar from fruits, plant materials, jello, carbonated beverages, caffeinated beverages, specialized flavor formulations emulating the taste of existing beers, wines and spirits, non-alcoholic cocktails, dealcoholized beer, dealcoholized wine, dealcoholized spirit, malt beer, tonic water, water and an alcoholic base beverage.

3. The alcoholic food product of claim 1, being an alcoholic beverage comprising a base alcoholic liquid, and at least one of a neurotransmitter or a neurotransmitter precursor.

4. The alcoholic food product of claim 3, wherein the base alcoholic beverage is selected from the group consisting of beer, wine, fruit wine, spirit, cider, perry and alcopop, wherein:
(a) the beer is at least one of ale, stout, porter, or lager;
(b) the wine is at least one of dry red wine, dry white wine, semi-dry red wine, semi-dry white wine, rose wine, dessert wine, Port wine, Champagne, sparkling wine or vermouth;
(c) the spirit is at least one of brandy, liquor, saki, Ouzo, arrack, rum, vodka, tequila, schnapps, whiskey, gin, cordial, Cachaca or slivovitz;
(d) the alcopop is at least one of beer cooler, wine cooler or enrich spirit cooler; or
(e) the fruit wine is at least one of grapefruit, orange, apple, berries, lemon, peach, apricot, plum, pomegranate, fig, dates, mango, melon, water melon, pear, guava, pineapple, passion fruit, kiwi, banana, litchi or papaya wine.

5. The alcoholic food product of claim 1, wherein the amount of alcohol is at least one of:
(a) from about 0.5% to about 98% by volume or by weight;
(b) from about 1% to about 20% by volume or by weight;
(c) from about 5% to about 10% by volume or by weight;
(d) from about 8% to about 12% by volume or by weight;
(e) from about 10% to about 15% by volume or by weight;
(f) from about 15% to about 25% by volume or by weight; or
(g) from about 30% to about 50% by volume or by weight.

6. The alcoholic food product of claim 1, wherein the neurotransmitter is at least one of norepinephrine, epinephrine, serotonin, dopamine, endorphin, acetylcholine, or a gamma-aminobutyric acid (GABA), and the neurotransmitter precursor is at least one of a norepinephrine precursor, an epinephrine precursor, a serotonin precursor, a dopamine precursor, an endorphin precursor, an acetylcholine precursor, or a gamma-aminobutyric acid (GABA).

7. The alcoholic food product of claim 6, wherein the neurotransmitter precursor is at least one dopamine precursor selected from the group consisting of L-phenylalanine, L-tyrosine, and levodopa.

8. The alcoholic food product of claim 7, wherein the neurotransmitter precursor is tyrosine.

9. The alcoholic food product of claim 8, wherein the amount of tyrosine is at least one of:
(a) in the range of from about 100 mg/L to about 5000 mg/L;
(b) in the range of from about 100 mg/L to about 1000 mg/L;
(c) about 350 mg/L;
(d) about 500 mg/L; or
(e) about 750 mg/L.

10. The alcoholic food product of claim 1, further comprising a psychostimulant substance which is at least one of caffeine, omega-3 fatty acids, magnesium, soluble fibers, folate, olive oil or monounsaturated fats extracted therefrom, green tea or theanine extracted therefrom, pregnenolone or a derivative thereof, uridine-5w-monophosphate, iron, turmeric or curcumin extracted therefrom, oregano or an extract thereof, Rhodiola rosea or an extract thereof, vitamin C, vitamin B6, or carbidopa.

11. The alcoholic food product of claim 10, wherein the psychostimulant substances is caffeine.

12. The alcoholic food product of claim 11, wherein the amount of caffeine is at least one of:
(a) from about 10 mg/L to about 900 mg/L;
(b) from about 10 mg/L to about 750 mg/L;
(c) about 35 mg/L;
(d) about 50 mg/L;
(e) about 100 mg/L; or
(f) about 350 mg/L.

13. The alcoholic food product claim 1, which upon consumption thereof exerts euphoric sensation which lasts from 5 minutes up to 24 hours after consumption.

14. The alcoholic beverage of claim 4, wherein the beer, wine, fruit wine, spirit, alcopop or cider comprises one or more neurotransmitter precursors in an amount that impart to the beverage the ability to exert positive psychoactive and psychotropic effects which exceed the effects exerted by a corresponding beer, wine, fruit wine, spirit, alcopop, and cider, respectively, that do not comprise a neurotransmitter precursor, while substantially minimizing intoxication effects.

15. An alcoholic beverage comprising a fermented base material, from about 3% to about 21% alcohol by volume, up to about 140 gr/L sugar, from about 200 mg/L to about 500 mg/L of at least one neurotransmitter precursor, from about 50 mg/L to about 200 mg/L of at least one psychostimulant substance, and flavorants.

16. The alcoholic beverage of claim 15, comprising at least one of:
(a) a fermented base material which is a fruit wine produced from at least one of:
natural or artificially flavored fruit juice, berry juice, fruit syrup, or fruit concentrate;
(b) alcohol in an amount of 10% by volume;
(c) tyrosine in an amount of from about 350 mg/L to about 450 mg/L;
(d) caffeine in an amount of from about 140 mg/L to about 150 mg/L; or
(e) a flavorant having a flavor which is at least one of bitter lime, lemon, red grapefruit or mint.

17. The alcoholic beverage of claim 15, wherein all the alcohol in the beverage is produced by fermentation of the fermentable base material.

18. The alcoholic beverage of claim 15, wherein the fermented base material is a fruit wine.

19. The alcoholic beverage of claim 18, wherein the fruit wine is apple wine.

20. An alcoholic beverage comprising from about 85% to about 95% apple wine by volume, from about 8% to about 11% undistilled alcohol by volume, from about 45 gr/L to about 55 gr/L sugar, from about 200 mg/L to about 500 mg/L tyrosine, from about 140 mg/L to about 150 mg/L caffeine, and a flavorant having a flavor selected from bitter lime, lemon, grapefruit, mint, grape, orange, ginger, apple, anise, a berry, lemon grass, peach, apricot, plum, pomegranate, fig, dates, mango, melon, water melon, pear, guava, pineapple, passion fruit, kiwi, banana, litchi, papaya, and any combination thereof.
